(19) United States
(12) Patent Application Publication
(10) Pub. No.: US 2025/0206872 A1
(43) Pub. Date: Jun. 26, 2025

(54) BIODEGRADABLE THERMOPLASTIC POLY(ORTHO ESTER) BASED MULTIBLOCK COPOLYMERS

(71) Applicants: INNOCORE TECHNOLOGIES HOLDING B.V., Groningen (NL); POLYVATION B.V., Groningen (NL)

(72) Inventors: Rob STEENDAM, Groningen (NL); Hendrik Jan HAITJEMA, Dedemsvaart (NL); Hajime NAKAJIMA, Kobe (JP); Albert DOORNBOS, Groningen (NL); Dirk PIJPER, Winschoten (NL); Leonardus Joannes VAN DER AA, Peize (NL); Theodorus Adrianus Cornelius FLIPSEN, Midlaren (NL)

(73) Assignees: INNOCORE TECHNOLOGIES HOLDING B.V., Groningen (NL); POLYVATION B.V., Groningen (NL)

(21) Appl. No.: 18/848,058

(22) PCT Filed: Mar. 17, 2023
(86) PCT No.: PCT/NL2023/050140

§ 371 Date: Sep. 17, 2024

(30) Foreign Application Priority Data
Mar. 17, 2022 (EP) 22162674.0

(51) Int. Cl. C08G 18/42; A61K 9/00; A61K 9/107; A61K 9/16; A61K 47/34; C08G 63/08; C08G 63/16; C08G 65/08

(52) U.S. Cl. CPC C08G 18/4252; A61K 9/0024; A61K 9/1075; A61K 9/1647; A61K 47/34; C08G 63/08; C08G 63/16; C08G 65/08; C08G 2230/00; C08G 2650/42

(57) ABSTRACT

The invention is directed to a biodegradable, thermoplastic multiblock copolymer, to a process for preparing a biodegradable, thermoplastic multiblock copolymer, to the use of a biodegradable thermoplastic multiblock copolymer, to a composition for the delivery of at least one biologically active compound to a host, and to a medical device comprising a biodegradable, thermoplastic multiblock copolymer. The biodegradable, thermoplastic multiblock copolymers of the invention comprise at least one prepolymer (A) segment and at least one hydrolysable amorphous prepolymer (B) segment, wherein the segments are linked by a multifunctional chain extender, wherein the prepolymer (A) segment:
a) comprises one or more hydrolysable linkages, and/or
b) comprises a water soluble polymer; and
   wherein the hydrolysable amorphous prepolymer (B) segment comprises a specified poly(ortho ester) block.

### BACKGROUND OF THE INVENTION

The invention is directed to a biodegradable, thermoplastic multiblock copolymer, to a process for preparing a biodegradable, thermoplastic multiblock copolymer, to the use of a biodegradable thermoplastic multiblock copolymer, to a composition for the delivery of at least one biologically active compound to a host, and to a medical device comprising a biodegradable, thermoplastic multiblock copolymer.

Peptides and proteins, together called polypeptides, play a vital role in all biological processes and have received a growing attention in recent years as drug candidates. The rapid advances in peptide and protein pharmacology along with the large-scale production of these compounds by recombinant DNA technology—among other techniques—have fuelled enormous interest in these compounds. Unfortunately, peptide and protein development has far outpaced the ability to deliver these compounds systemically or locally using convenient and effective delivery systems.

Biodegradable polymers have received increased attention over the past decade for use in long-acting parenteral controlled release systems, either for systemic or site-specific drug delivery. Biodegradable controlled release formulations can significantly improve the pharmacokinetics of therapeutic compounds. This is especially relevant in the treatment of chronic diseases and for compounds with a narrow therapeutic window since systemic plasma concentrations can be reduced with concurrent reduction in undesirable side effects. Additionally, many new biologically active compounds have short half-lives, necessitating frequent injection to achieve therapeutically effective plasma levels. Patient compliance and the high costs associated with frequent dosing regimens for parenterally administered biologically active compounds have increased the interest in biodegradable sustained release dosage forms.

Poly(D,L-lactic acid) (PDLLA) and copolymers of lactic acid and glycolic acid (PLGA), are the most widely applied biodegradable polymers for use in parenteral sustained release depot formulations. These polymers have the advantage that they have a proven track record of clinical use, are generally considered as highly biocompatible and degrade into non-toxic degradation products that are metabolised and/or excreted from the body via known pathways. As a result, PLGA, PDLLA and related (co-) polymers have been adopted and successfully used by pharmaceutical companies for the development of long-acting injectable and implantable depot formulations for small molecules, such as risperidone (Risperdal® Consta), dexamethasone (Ozurdex®) and triamcinolone acetonide (Zilretta®), and therapeutic peptides such as leuprolide (Lupron® Depot), goserelin (Zoladex®) and octreotide (Sandostatin® LAR). PLGA polymers however exhibit certain physicochemical and degradation characteristics that limit their use and make them less suitable for sustained release delivery of polypeptides. Firstly, PLGA copolymers are relatively hydrophobic and as a result do not provide an optimal environment for hydrophilic or amphiphilic polypeptides. Polypeptides may adsorb to the polymer, resulting in slow and incomplete release, structural unfolding and/or aggregation. Secondly, the ability to manipulate the release of encapsulated polypeptides, and especially larger polypeptides such as (recombinant) proteins, growth factors or (monoclonal) antibodies, is limited since diffusion of such polypeptides through the relatively rigid, hydrophobic and non-swellable PLGA matrices is negligible. The release of polypeptides from PLGA copolymers therefore depends on diffusion via pores present in the matrix and on the degradation of the polymer matrix. Typically, the encapsulated polypeptide remains entrapped in the PLGA matrix until the moment the latter has degraded to such an extent that it loses its integrity or dissolves, resulting in biphasic or triphasic degradation-dependent release typically observed for PLGA-based extended release polypeptide formulations. Finally, during degradation of PLGA copolymers, acidic moieties are formed that accumulate in the rigid and non-swellable PLGA matrix resulting in the formation of an acidic micro-environment in the polymer matrix with in situ pHs that can be as low as 1-2. Such acidic conditions may have a deleterious effect on the structural integrity and biological activity of the encapsulated polypeptide, potentially leading to reduced therapeutic efficacy or side effects. The encapsulated polypeptides may form aggregates leading to incomplete release and enhanced immunogenicity. Moreover, the polypeptide may be chemically modified. Peptide acylation and adduct formation have been reported for PLGA-based extended release polypeptide formulations such as Sandostatin® LAR extended release octreotide microparticles (Ghassemi et al., Pharm. Res. 2012, 29(1), 110-20).

The limitations of PLGA-based amorphous polymers regarding the delivery of polypeptides can be resolved by using biodegradable polyester-based polymers containing a hydrophilic water-swellable polymer moiety in their structure. WO-A-2005/068533 describes amorphous polyether ester multiblock copolymers containing water soluble polyethylene glycol (PEG) units. Due to their low glass transition temperature (T_{g}) of <37° C. under physiological conditions, these amorphous multiblock copolymers are permeable for both low molecular weight polypeptides such as leuprolide as well as for the acidic degradation products formed upon hydrolysis of the polymer. As a consequence, gradual release of such polypeptides as well as the (acidic) degradation products that are being formed can be achieved—thereby preventing the accumulation of acidic degradation products in the polymer matrix and the formation of an acidic microenvironment.

Despite their usefulness for sustained release of small molecule drugs and low molecular weight peptides, the amorphous multiblock copolymers disclosed in WO-A-2005/068533 are not suitable for the sustained release of larger polypeptides such as recombinant proteins and monoclonal antibodies. This is due to the fact that high weight fractions of water soluble PEG would need to be introduced into such amorphous multiblock copolymers as to create a polymer matrix with sufficient swelling degree to allow diffusion-controlled release of the encapsulated large polypeptide. However, the incorporation of large weight fractions of PEG in the structure of such an amorphous multiblock copolymer dramatically reduces its T_{g} to values below room temperature or even below 0° C. Amorphous multiblock copolymers with such a low T_{g} typically suffer from processability issues (sticky polymers) and cannot be processed into solid drug delivery formulations, such as microspheres or implants. Furthermore, there is a high chance that extended release drug delivery products derived from such low T_{g} amorphous multiblock copolymers will suffer from stability issues and have insufficient shelf life when stored under ambient or refrigerated conditions.

To overcome the shortcomings encountered with the amorphous multiblock copolymers, the inventors developed biodegradable phase separated thermoplastic segmented multiblock copolymers based on a crystalline poly(ε-caprolactone) block (as disclosed in WO-A-2012/005594) or a crystalline poly(L-lactide) block (as disclosed in WO-A-2013/015685) in combination with a PEG containing hydrophilic block. Such multiblock copolymers allow the preparation of depot formulations with long-term sustained release of structurally intact and biologically active polypeptides over extended period of time.

Hydrophilic phase separated segmented multiblock copolymers containing a hydrophobic poly(ε-caprolactone)-based crystalline block, as disclosed in WO-A-2012/005594, were found to be well processable into implants by hot melt extrusion and allowed long-term sustained release of peptides and proteins (Stankovic et al., Eur. J. Pharm. Sci. 2013, 49(4), 578-587). Hydrophilic phase separated segmented multiblock copolymers containing a hydrophobic poly(L-lactide)-based crystalline block, as disclosed in WO-A-2013/015685, were shown to have highly beneficial attributes in regard to protein delivery. Especially multiblock copolymers composed of a poly(ε-Caprolactone)-PEG-poly(ε-Caprolactone)-based hydrophilic block in combination with a poly(L-lactide)-based crystalline block (PCL multiblock copolymers) were found to exhibit promising characteristics allowing long-term sustained release of structurally intact biologics when formulated into microparticles (Teekamp et al., Int. J. Pharm. 2017, 534(1-2), 229-236; Teekamp et al., J. Control Release 2018, 269, 258-265; Scheiner et al., ACS Omega 2019, 4(7), 11481-11492).

The authors further found that the biodegradable, phase separated, thermoplastic multiblock copolymers containing a poly(L-lactide) crystalline block have a degradation time of 3-4 years. Multiblock copolymers containing a poly(ε-caprolactone) crystalline block are expected to have an even longer degradation time. For the majority of sustained release drug delivery formulations, such a degradation time is unacceptably long as it would lead to polymer accumulation upon repeated injection and could potentially induce long-term tolerability issues. It would be desirable to have a multiblock copolymer with a shorter degradation time, such as a degradation time of approximately 0.5-1.5 years, depending on the duration of release. In addition, due to their high melting temperature (120-140° C.), most of the semi-crystalline poly(L-lactide)-based multiblock copolymers disclosed in WO-A-2013/015685 require high temperatures for processing into implants via hot melt extrusion, injection moulding or 3D printing, which could lead to thermal-stress induced degradation of the incorporated drug. Finally, the hardening of microspheres prepared of semi-crystalline poly(L-lactide)-based multiblock copolymers via solvent extraction/evaporation-based emulsification processes or spray-drying strongly depends on the crystallisation rate of the semi-crystalline block. Multiple factors such as polymer concentration, rate of solvent removal, type of active pharmaceutical ingredient or temperature, strongly affect the crystallisation rate of the polymer during microsphere production, and thus small changes in those parameters can drastically impact the hardening of the microspheres. As a consequence, the production of microspheres using such polymers is accompanied with challenges regarding reproducibility, scaling up and storage stability. At the same time, it would be beneficial to retain the excellent tunability of the drug release kinetics reported for poly(L-lactide) and poly(ε-caprolactone)-based multiblock copolymers disclosed in WO-A-2013/015685 and WO-A-2012/005594.

The above-mentioned drawbacks regarding the use of semi-crystalline polymers can be overcome by using amorphous polymers for preparation of long-acting injectable formulations. However, as described above, the typical amorphous biodegradable polyesters, including the amorphous multiblock copolymers disclosed in WO-A-2005/068533, that allow sustained release of polypeptides do not have the required thermomechanical characteristics to obtain solid and stable long-acting injectable formulations.

A redesign of SynBiosys® PCL multiblock copolymers was conducted in an attempt to reduce the erosion time of the polymer as to avoid polymer accumulation upon repeated administration and improve the long-term local tolerability. To overcome polymer-crystallisation-related reproducibility and stability issues, an amorphous prepolymer (B) was designed to replace the crystalline poly(L-lactide) and poly(ε-caprolactone)-based prepolymer (B) segments disclosed in WO-A-2013/015685 and WO-A-2012/005594.

However, to obtain a multiblock copolymer that has a sufficiently high T_{g} to be processed into solid drug delivery formulations that are stable under ambient storage conditions, any amorphous prepolymer (B) segment to be considered to replace a crystalline prepolymer (B) segment such as poly(L-lactide) should exhibit a sufficiently high T_{g} as to compensate for a low T_{g} prepolymer segment (A)—such as a prepolymer (A) segment containing relatively large amounts of ε-caprolactone or polyethylene glycol—and in case phase mixing of amorphous prepolymer (A) segment with the amorphous prepolymer (B) segment occurs as reported for amorphous multiblock copolymers disclosed in WO-A-2005/068533. The prepolymer blocks used in the amorphous multiblock copolymers in WO-A-2005/068533 have relatively low T_{g}s due to which the resulting amorphous multiblock copolymers exhibit T_{g} values varying from −24° C. to 21.4° C., which is typically too low to obtain drug products that are stable during storage at room temperature or under refrigerated conditions. Preferably, amorphous polymers used in drug delivery products should have a T_{g} significantly exceeding the intended storage conditions, as to minimise polymer chain relaxation thereby preventing migration of the incorporated active compound in the polymer matrix. Preferably such amorphous polymers should have a T_{g} of around 40° C. or higher.

Thus, there remains a need for biodegradable polymers that can overcome the one or more of the shortcomings of prior art polymers and that are more suitable for the delivery of polypeptides. More specifically such polymers should preferably be (i) amorphous and have a sufficiently high glass transition temperature to assure product stability under the desired storage conditions, (ii) be compatible with polypeptides and allow sustained release of intact and biologically functional polypeptides, and (iii) have acceptable erosion kinetics. It is furthermore desired that such a new biodegradable delivery system for polypeptides would be designed of prepolymers that are composed of monomers that are well-known, biologically safe and clinically acceptable.

Poly(ortho ester)s are used as biodegradable polymeric excipients in injectable and implantable sustained release drug delivery products. Contrary to poly(L-lactide) based polymers, poly(ortho ester)s are amorphous. They are typically prepared by the reaction of 3,9-diethylidene-2,4,8,10-tetraoxaspiro [5.5]undecane (DETOSU) with a diol. Due to their hydrophobic character they degrade very slowly under physiological conditions. To speed up their degradation and to make them suitable for human and veterinary drug delivery applications, short latent acid segments based on glycolic or lactic acid were incorporated in the structure of DETOSU-based poly(ortho ester)s (Ng et al., Macromolecules 1997, 30(4), 770-772; U.S. Pat. No. 5,968,543). Upon hydrolysis of the latent acid segment, glycolic or lactic acid is formed which then catalyses the hydrolysis of the ortho ester units in the polymer chain. The degradation rate of such poly(ortho ester)s can be tuned by adjusting the latent acid content inside the polymer backbone. Next to the degradation rate, the thermal and mechanical properties of poly(ortho ester)s can be altered by changing the type and content of the diol in the structure of the poly(ortho ester)s (U.S. Pat. No. 5,968,543). The replacement of rigid diols by more flexible analogues leads to materials with lower T_{g} and wax-like properties. The structure of poly(ortho ester)s can be further diversified by the addition of hydrophilic diols (triethylene glycol and PEG) to the reaction mixture. The presence of more hydrophilic diols leads to higher water uptake and consequently faster degrading polymers (U.S. Pat. No. 5,968,543; WO-A-2006/105148; Ng et al., J. Controlled Release 2000, 65(3), 367-374) making them suitable matrices for the preparation of sustained release formulations in the form of microspheres, implants, films, ointments, or in situ forming implants for the delivery of either small molecular weight drugs and big molecules as proteins and DNA molecules. Ng et al. (J. Controlled Release 2000, 65(3), 367-374) demonstrated tuneable release of 5-fluorouracil from poly(ortho ester) films by varying the content of both hydrophilic triethylene glycol and glycolide units in the poly(ortho ester) backbone. Similarly, the incorporation of small amounts of PEG in the poly(ortho ester) was used to speed up the release and reduce the lag phase in the release of bovine serum albumin (Rothen-Weinholod et al., J. Controlled Release 2001, 71(1), 31-37). Wang et al. (Nature Materials 2004, 4(3), 190-196) illustrated pH tuneable release of DNA plasmid from polymer microspheres by incorporating small amounts of tertiary amine units (methyl diethanolamine) in the polymer backbone. In this way, prolonged release of DNA at low pH was obtained compared to the polymer without tertiary amine units.

Their tuneable degradation and good biocompatibility granted this group of materials good results during clinical investigation and resulted in few products released on the market (Sustol® and Zynrelef). Currently marketed poly(ortho ester)s based drug delivery products are primarily used in the form of small molecular weight semi-solid materials with or without added biocompatible solvent (WO-A-2014/143635; Heller et al., J. Controlled Release 2002, 78(1-3), 133-141).

US-A-2014/0 113 975 discloses biodegradable AB diblock and BAB or ABA triblock copolymers of relatively low molecular weight for application as a flowable liquid drug delivery systems for improved drug delivery. The copolymers of US-A-2014/0 113 975 comprise a PEG, a PEG derivative or a mixture of PEG and PEG derivative, and a biodegradable ABA, BAB and AB type block copolymers that are based on biodegradable hydrophobic polyester or poly(ortho ester) A blocks and PEG B blocks. According to US-A-2014/0 113 975, high molecular weight polyester synthesis of such ABA, BAB and AB type block copolymers is possible by ring opening polymerisation, but high molecular weight poly(ortho ester)s A blocks cannot be obtained following the addition reaction-based synthesis route used in the examples 12 and 13 of US-A-2014/0 113 975. Due to the nature of the addition reaction used for the synthesis of the poly(ortho ester) A blocks, it is challenging to reproducibly prepare polymers of well controlled high molecular weight along with tunable hydrophobic/hydrophilic block composition, which is a serious limitation. In particular, the triblock copolymers of USA2014/0 113 975 are limited to copolymers where the hydrophilic block is either in the middle (ABA) or in the periphery (BAB).

The main advantage of poly(ortho ester)s over other amorphous polymer systems is the possibility of preparing materials with a T_{g} as high as 90° C. However, the incorporation of acidic moieties, hydrophilic aliphatic diols or PEG units, which is crucial as to obtain polymers with acceptable degradation kinetics, can significantly reduce the T_{g} to a values below room temperature. For example, the swellable and/or fast degrading poly(ortho ester)s described in U.S. Pat. No. 5,968,543, WO-A-2006/105148 and US-A-2014/0 113 975 are lacking a sufficiently high T_{g} to allow long-term storage under ambient conditions of pre-formed drug delivery products, such as microparticle- or solid implant-based drug delivery products.

The physicochemical properties of polymers can be strongly affected by the monomer distribution within copolymer. Poly(ortho ester)s described in U.S. Pat. No. 5,968,543 and WO-A-2006/105148 are prepared by reacting diketene acetals with diols. Such a reaction mechanism results in a random distribution of the diol units in the polymer backbone. Due to the random distribution of the diol units, the properties of the polymer cannot be controlled which is a significant drawback when further optimisation of drug delivery formulations prepared of such polymers is required. It would be highly preferred to have control over the distribution of the acidic and/or hydrophilic moieties in the polymer backbone, such as to have the moieties incorporated in a more blocky manner, as this would provide a wider and more versatile toolkit for customisation of the polymer characteristics and improvement of the drug release and degradation kinetics

Furthermore, the synthesis of poly(ortho ester)s comprises a step growth reaction and requires the presence of diols in a reaction mixture. The nature of the step growth reaction limits the type of functionalities that can be incorporated in the main chain. For instance, the incorporation of poly(amino acids)-based segments in the poly(ortho ester) backbone, that can add extra functionality to the polymer and improve interaction of the polymer with proteins and peptides, can be a difficult task to achieve. The preparation of poly(amino acid) diols meeting the requirements for the synthesis of poly(ortho esters) described in U.S. Pat. No. 5,968,543 and WO-A-006/105148 requires multiple synthesis steps which can potentially lead to an increase of the number and level of impurities in the final poly(ortho ester)s.

The invention is directed to multiblock copolymers that comprise at least two prepolymer segments with one of the segments containing a short poly(ortho ester) block. The multiblock copolymers of this invention are preferably amorphous in the dry state. More in particular, a copolymer according to the invention is a biodegradable multiblock copolymer, wherein at least two prepolymer segments are linked by multifunctional chain extender. The multifunctional chain extender is preferably an aliphatic chain extender.

When compared to prior art semi-crystalline polymers, the polymers of the invention degrade faster and their degradation can be easily tuned by changing the composition of the block used in combination with the poly(ortho ester) block in the multiblock copolymer. Moreover, since they are composed of an amorphous poly(ortho ester) block, crystallisation-related processability, reproducibility and storage stability problems can be avoided.

In contrast to PLGA and PDLLA polymers or the multiblock copolymers disclosed in WO-A-2005/068533, which degrade via bulk erosion, the polymers of the invention degrade primarily via surface erosion. This has as advantage that more gradual mass loss occurs and more constant drug release kinetics can be obtained. In addition, polymers disclosed in WO-A-2005/068533 undergo hydrolysis under both basic and acidic conditions. This portrays as a significant challenge when the encapsulated drug is highly basic, such as for example risperidone. Basic nucleophilic (drug) molecules can catalyse the hydrolysis of the ester bonds in hydrated or dissolved state and cause degradation of polymers during processing or storage (Wang et al., Adv. Drug Deliv. Rev. 2021, 178, 113912). As a result, due to which critical characteristics, such as for example drug release kinetics, can be affected. Contrary to polymers disclosed in WO-A-2005/068533 or commonly used PLGA polymers, poly(ortho ester) blocks are stable against hydrolysis under basic conditions and degradation of the polymer is prevented when basic (drug) molecules are encapsulated in poly(ortho ester)-based polymer matrix. In addition, due to the high hydrophobicity of poly(ortho ester) blocks, water penetration into the polymer matrix is slow and limited, thereby preventing bulk hydrolysis of the polymer matrix. Instead the poly(ortho ester) based polymer matrix exhibits surface erosion behaviour thereby potentially allowing more gradual surface-erosion controlled release of the encapsulated drug molecule. In this way the incorporation of poly(ortho ester) blocks increases the stability of biodegradable thermoplastic multiblock copolymers in the presence of a highly basic drug molecule and longer and more constant release of such a drug molecule is easier to achieve.

Contrary to the polymers disclosed in U.S. Pat. No. 5,968,543 and WO-A-2006/105148, where acid or hydrophilic moieties are distributed randomly within the polymer backbone, in the polymers of the invention the acidic or hydrophilic moieties are introduced as blocks comprising multiple acidic and/or hydrophilic moieties and as a consequence are distributed in a more blocky manner. Multiblock copolymers composed of (pre-made) blocks of different composition and with different physicochemical characteristics are more likely to phase separate. With such a structure biphasic release patterns can be achieved. For example, if one of the segments has low permeability and/or is slowly degrading and the other segment has high permeability and/or degrades rapidly, encapsulated drug molecules will initially be released predominantly from the phase which has a high permeability and/or degrades rapidly, before release of drug molecules encapsulated in the phase with lower permeability/degradation rate will start to occur. By modifying the permeability and/or degradation rates of the two phases, the rate of release of a drug from a specific phase can be controlled. In addition, contrary to random orientation of monomer units inside the polymer, more blocky structures have a higher chance of phase separating. It is known that above a certain block length, block copolymers can undergo phase separation, whereby each block keeps its own properties, such as T_{g} or degradation rate. Thus, the polymers of the invention can still exhibit good structural integrity, since due to phase separation between the high T_{g} poly(ortho ester) block and any other low T_{g} polymer block, the relatively high T_{g} of the poly(ortho ester) block is not significantly decreased.

Furthermore, the polymers of the invention are prepared by a chain extension reaction. In contrast to the synthetic procedure required for the preparation of polymers disclosed in U.S. Pat. No. 5,968,543 and WO-A-2006/105148, such a chain extension reaction is not limited to the use of diols in the reaction mixture. Next to diol-terminated short polymer blocks, other types of difunctional blocks, such as diacids or diamines can be used, as long as they fulfil the requirement that they can react with the multifunctional chain extender.

### SUMMARY OF THE INVENTION

In a first aspect, the invention is directed to a biodegradable, thermoplastic multiblock copolymer, comprising at least one prepolymer (A) segment and at least one hydrolysable amorphous prepolymer (B) segment, wherein the segments are linked by a multifunctional chain extender, wherein the prepolymer (A) segment:
a) comprises one or more hydrolysable linkages, and/or
b) comprises a water soluble polymer; and
wherein the hydrolysable amorphous prepolymer (B) segment comprises the following structure:

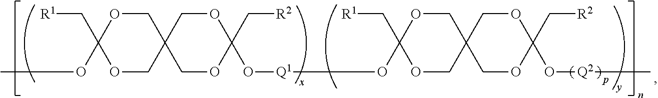

wherein
n is 4-100, such as 5-50,
x is 0.25-1 and x+y=1,
p is 0 or 1;
R¹ and R² are independently selected from hydrogen and C₁-C₄ alkyl;
Q¹ is selected from

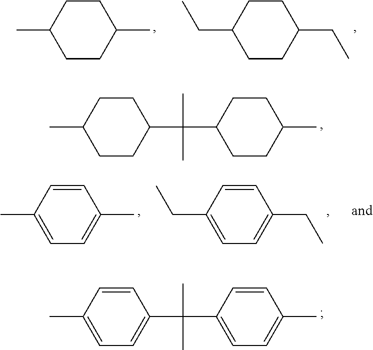

Q² is selected from

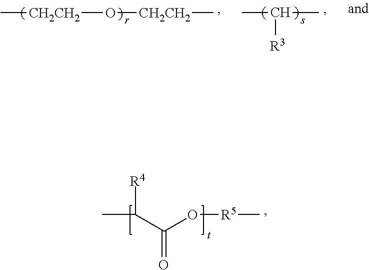

wherein
r is 1-100,
s is 1-12,
t is 1-10,
R³ is selected from hydrogen and C₁-C₆ alkyl,
R⁴ is selected from hydrogen and C₁-C₄ alkyl, and
R⁵ is selected from

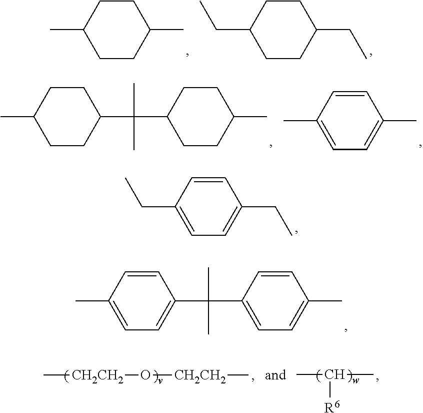

wherein
   v is 1-100,
   w is 1-12, and
   R⁶ is selected from hydrogen and C₁-C₆ alkyl.

In a further aspect, the invention is directed to a process for preparing a biodegradable, thermoplastic multiblock copolymer according to the invention, comprising a chain-extension reaction of prepolymer (A) and prepolymer (B) in the presence of a multifunctional chain extender.

In yet a further aspect, the invention is directed to a composition for delivery of at least one biologically active compound to a host, comprising at least one biologically active compound encapsulated in a matrix, wherein said matrix comprises at least one biodegradable, thermoplastic multiblock copolymer according to the invention.

In yet a further aspect, the invention is directed to a medical device in the form of microspheres, microparticles, nanoparticles, nanospheres, rods, solid implants, gels, in situ forming implants, coatings, films, sheets, sprays, tubes, membranes, meshes, fibres, scaffolds or plugs, wherein said medical device comprises a biodegradable, thermoplastic multiblock copolymer of the invention.

### BRIEF DESCRIPTION OF THE DRAWINGS

FIG. 1 In vitro erosion of polymer-only microspheres composed of 50CP10C20-LL40: experimental data up to 12 months and extrapolation of the experimental data up to complete erosion.

FIG. 2 Molecular structure of diol-functionalised poly(ortho ester) prepolymer derived from DVTOSU and CHDM.

FIG. 3 Reaction scheme for preparation of 20CP10C20-POE40.

FIG. 4 SEM images of polymer-only microspheres prepared of 20CP10C20-POE40 (RCP 1679).

FIG. 5 In vitro erosion of polymer-only microspheres composed of 50CP10C20-LL40, 20CP10C20-POE40 and 50CP10C20-POE40.

FIG. 6 Cumulative in vitro release of protein ICP002 from microspheres prepared of 20CP10C20-POE40/50CP10C20-POE40 blends with blend ratios of 0/100 (PBD17-008), 33/67 (PBD17-027), 50/50 (PBD17-025), 67/33 (PBD17-024) and 100/0 (PBD17-011). Release is shown as μg ICP002 released in time.

FIG. 7 Reaction scheme for preparation of 20LP6L12-POE40.

FIG. 8 Reaction scheme for preparation of 10L40-POE40.

FIG. 9 Reaction scheme for preparation of 20GL40-POE40.

FIG. 10 In vitro erosion of polymer-only microspheres composed of 100POE40, 10L40-POE40, 50L40-POE40, 25GL40-POE40, and 50LP10L20-POE40.

FIG. 11 SEM images of ropivacaine loaded microspheres prepared of 20L40-POE40 (210554), 20GL40-POE40 (210555), 50L40-POE40 (210556) and 20LP6L12-POE40 (210557).

FIG. 12 Cumulative in vitro release of ropivacaine from microspheres prepared of 20L40-POE40, 20GL40-POE40, 50L40-POE40 and 20LP6L12-POE40.

FIG. 13 Cumulative in vitro release of ropivacaine from NMP based in situ forming implants composed of 20L40-POE40, 20GL40-POE40, 50L40-POE40 and 20LP6L12-POE40.

FIG. 14 Cumulative in vitro release of levonorgestrel from NMP based in situ forming implants composed of 10L40-POE40, 20L40-POE40, 20GL40-POE40, 50L40-POE40 and 20LP6L12-POE40.

FIG. 15 Cumulative in vitro release of leuprolide from NMP/BB 90/10 w/w based in situ forming implants composed of 50L40-POE40, 20L40-POE40 and 20GL40-POE40.

### DETAILED DESCRIPTION OF THE INVENTION

The term “prepolymer” as used herein is meant to refer to the polymer segments that are linked by a multifunctional chain extender, together making up the multiblock copolymer of the invention. Each prepolymer may be obtained by polymerisation of suitable monomers, which monomers thus are the chemical units of each prepolymer. The properties of the prepolymers and, by consequence, of the multiblock copolymer of the invention, can be controlled, amongst others, by choosing a prepolymer of a suitable composition and molecular weight.

The terms “block” and “segment” as used herein are meant to refer to distinct regions in a multiblock copolymer. The terms block and segment are used interchangeably.

The term “multiblock” as used herein is meant to refer to the presence of at least two distinct prepolymer segments in a polymer chain.

The term “thermoplastic” as used herein is meant to refer to the non-crosslinked nature of the multiblock copolymer. Upon heating, a thermoplastic polymer becomes fluid, whereas it solidifies upon (re-)cooling. Thermoplastic polymers are soluble in proper solvents.

The term “hydrolysable” as used herein is meant to refer to the ability of reacting with water upon which the molecule is cleaved. Hydrolysable segments, for instance, include esters, carbonates, anhydrides, amides, phosphates, phosphazenes, urethanes, and ureas. The multiblock copolymers of the invention can comprise, e.g., one or more hydrolysable linkages selected from the group consisting of enol ether, acyclic acetal, anhydride, carbonate, phosphazene, N-substituted amido, N-substituted urethane, N-substituted imino, imido, substituted imido, N,N-disubstituted hydrazo, thioester, phosphonic ester, sulphonic ester, ortho ester, ether, thio and siloxyl linkages.

The term “multifunctional chain extender” as used herein is meant to refer to the presence of at least two reactive groups on the chain extender that allow chemically linking reactive prepolymers thereby forming a multiblock copolymer.

The term “water-soluble polymer” as used herein is meant to refer to a polymer that has a good solubility in an aqueous medium, such as water, under physiological conditions. This polymer, when copolymerised with more hydrophobic moieties, renders the resulting copolymer swellable in water. The water-soluble polymer can be a diol, a diamine or a diacid. The diol or diacid is suitably used to initiate the ring-opening polymerisation of cyclic monomers. Typically, a water-soluble polymer will transmit at least 75%, more preferably at least 95%, of light transmitted by the same solution after filtering. On a weight basis, a water-soluble polymer will preferably be at least 35% soluble in water, more preferably at least 50%, still more preferably 70%, and still more preferably 85%. It is most preferred, however, that on a weight bases the water-soluble polymer is 95% soluble in water or completely soluble in water.

The term “swellable” as used herein is meant to refer to the uptake of water by the polymer. The swelling ratio can be calculated by dividing the mass of the water-swollen copolymer by that of the dry copolymer.

The term “biologically active compound” as used herein is intended to be broadly interpreted as any agent that provides a therapeutic or prophylactic effect. Such agents include, but are not limited to, antimicrobial agents (including antibacterial and antifungal agents), anti-viral agents, anti-tumour agents, hormones and immunogenic agents.

The term “biologically active polypeptide” as used herein is meant to refer to peptides and proteins that are biologically active in a mammal body, more in particular in the human body.

The inventors surprisingly found that the multiblock copolymers of the invention, comprising a prepolymer (A) segment with hydrolysable linkages and/or a water-soluble polymer and an amorphous prepolymer (B) segment with a specific poly(ortho ester) prepolymer block, have desirable properties which renders them suitable for sustained release of, for example, active pharmaceutical ingredients (including biologically active compounds such as small molecules, peptides or protein-based therapeutic agents). Without wishing to be bound by any theory, the inventors believe that the polymers of the invention degrade predominantly via a surface erosion mechanism leading to a more gradual degradation and release profile. The prepolymer (B) segment ensures a relatively high T_{g} and hydrophobicity of the multiblock copolymer so that penetration of water into the polymer matrix is slow and diffusion of active pharmaceutical ingredient from the inner (non-hydrated) part of the material is restricted or limited. At the surface, the polymer is in contact with surrounding water. Water may cause polymer degradation at the surface by hydrolysis of hydrolysable linkages in the prepolymer (A) and prepolymer (B) segments, thereby releasing active pharmaceutical ingredient from the surface region. Alternatively, or in addition, water may cause swelling of polymer at the surface via water-soluble polymer being present in the prepolymer (A) segment, thereby allowing faster diffusion and release of active pharmaceutical ingredient contained in the polymer from the surface region.

The multiblock copolymers of the invention comprise at least one hydrolysable prepolymer (A) segment and at least one hydrolysable prepolymer (B) segment that are linked by a multifunctional chain extender.

The prepolymer (A) segment comprises one or more hydrolysable linkages, and/or a water soluble polymer.

Examples of hydrolysable linkages include esters linkages, carbonate linkages, anhydride linkages, amide linkages, phosphate linkages, phosphazene linkages, urethane linkages, and urea linkages. The multiblock copolymers of the invention can comprise, e.g., one or more hydrolysable linkages selected from the group consisting of enol ether, acyclic acetal, anhydride, carbonate, phosphazene, N-substituted amido, N-substituted urethane, N-substituted imino, imido, substituted imido, N,N-disubstituted hydrazo, thioester, phosphonic ester, sulphonic ester, ortho ester, ether, thio and siloxyl linkages. In terms of time scale, ester, carbonate and/or phosphazene linkages are preferred.

Preferably, prepolymer (A) comprises reaction products of one or more cyclic monomers and/or one or more non-cyclic monomers.

Cyclic monomers can, for instance, be selected from the group consisting of glycolide, L-lactide, D-lactide, D,L-lactide, ε-caprolactone, δ-valerolactone, trimethylene carbonate, tetramethylenecarbonate, 1,5-dioxepane-2-one, 1,4-dioxane-2-one (p-dioxanone), cyclic anhydrides (such as oxepane-2,7-dione), N-carboxyanhydrides of natural amino acids and their derivatives (such as N-carboxyalanine anhydride) and morpholine-2,5-diones based cyclic depsipeptides (such as 6-methyl-morpholine-2,5-dione). Non-cyclic monomers can, for instance, be selected from the group consisting of succinic acid, glutaric acid, adipic acid, sebacic acid, lactic acid, glycolic acid, hydroxybutyric acid, natural amino acids and their derivates (such as alanine), ethylene glycol, diethylene glycol, 1,4-butanediol, 1,6-hexanediol, 1,4-butanediamine and 1,6-hexanediamine.

In case prepolymer (A) comprises poly(D,L-lactide), the L/D ratio of the lactide may be away from unity (other than 50/50). For instance, an L/D ratio between 85/15 and 15/85 gives a completely amorphous homopolymer. Furthermore, it is known that an excess of one isomer (L or D) over the other increases the T_{g} of the poly(D,L-lactide).

In case prepolymer (A) comprises poly(D,L-lactide-co-glycolide), the D,L-lactide/glycolide molar ratio may be away from unity (other than 50/50). For instance, a D,L-lactide/glycolide molar ratio >1 or <1, such as would be the case for a poly(D,L-lactide-co-glycolide) with a D,L-lactide/glycolide molar ratio of 85/15 or 15/85, will lead to slower degradation as compared to poly(D,L-lactide-co-glycolide) with a D,L-lactide/glycolide molar ratio of 50/50. Furthermore, an excess of one monomer (D,L-lactide or glycolide) over the other increases the T_{g} of the poly(D,L-lactide-co-glycolide).

Furthermore, prepolymer (A) can be based on (mixtures of) condensation (non-cyclic) type of monomers such as hydroxyacids (e.g. lactic acid, glycolic acid, hydroxybutyric acid), diacids (e.g. glutaric, adipic or succinic acid, sebacic acid) and diols such as ethylene glycol, diethylene glycol, 1,4-butanediol or 1,6-hexanediol, forming ester and/or anhydride hydrolysable moieties.

The prepolymer (A) segment can comprise a water-soluble polymer. This water-soluble polymer may comprise one or more polyethers (such as polyethylene glycol (PEG), polytetramethyleneoxide (PTMO), polypropyleneglycol (PPG), and polytetramethylene ether glycol (PTMG)), or one or more other water-soluble polymers (such as polyvinylalcohol (PVA), polyvinylpyrrolidone (PVP), polyvinylcaprolactam, poly(hydroxyethylmethacrylate) (poly-(HEMA)), or polyphosphazenes. The prepolymer (A) segment may also comprise blends and/or copolymers of two or more of these polymers. Preferably, the prepolymer (A) segment comprises a water-soluble polymer that is derived from poly(ethylene glycol) (PEG). Said poly(ethylene glycol) can, for example, have a number average molecular weight Mₙ of 150-10 000 g/mol, preferably 300-5000 g/mol, more preferably 600-3000 g/mol.

Some non-limiting examples of suitable prepolymer (A) segments include poly(D,L-lactide-co-glycolide), poly(D,L-lactide), poly(ε-caprolactone), poly(p-dioxanone), poly(D,L-lactide)-co-PEG-co-poly(D,L-lactide), poly(glycolide)-co-PEG-co-poly(glycolide), poly(ε-caprolactone)-co-PEG-co-poly(ε-caprolactone), and poly(p-dioxanone)-co-PEG-co-poly(p-dioxanone). It is also possible that prepolymer (A) segment does not have any hydrolysable linkages, but consists of one or more water-soluble polymers such as PEG, PTMO, PPG, PTMG, PVA, PVP, polyvinylcaprolactam, poly-(HEMA), polyphosphazenes, or combinations thereof.

In any addition, the prepolymer (A) segment can comprise a water-soluble polymer and have, at each side of the water-soluble polymer, any copolymer of the above-mentioned monomers. Some non-limiting examples of such prepolymer (A) segments include [poly(ε-caprolactone-co-D,L-lactide)]-co-PEG-co-[poly(ε-caprolactone-co-D,L-lactide)], [poly(ε-caprolactone-co-glycolide)]-co-PEG-co-[poly(ε-caprolactone-co-glycolide)], [poly(ε-caprolactone-co-p-dioxanone)]-co-PEG-co-[poly(ε-caprolactone-co-p-dioxanone)], [poly(D,L-lactide-co-glycolide)]-co-PEG-co-[poly(D,L-lactide-co-glycolide)], [poly(D,L-lactide-co-p-dioxanone)]-co-PEG-co-[poly(D,L-lactide-co-p-dioxanone)], and [poly(glycolide-co-p-dioxanone)]-co-PEG-co-[poly(glycolide-co-p-dioxanone)].

If the prepolymer (A) segment comprises a water-soluble polymer, then 10% or more by total weight of prepolymer (A) may consist of water-soluble polymer, such as 15% or more, 20% or more, 30% or more, 40% or more, 50% or more, 60% or more, or 70% or more. Suitably, 95% or less by total weight of prepolymer (A) may consist of water-soluble polymer, such as 90% or less, 85% or less.

Prepolymer (A) can have a number average molecular weight (Mₙ) of 300 g/mol or more, such as 500 g/mol or more, 1000 g/mol or more, 1500 g/mol or more, or 2000 g/mol or more. Prepolymer (A) can have a Mₙ of 30 000 g/mol or less, such as 20 000 g/mol or less, 10 000 g/mol or less, 8000 g/mol or less, 7000 g/mol or less, 5000 g/mol or less, 4000 g/mol or less, 3000 g/mol or less, or 2500 g/mol or less. The length of the prepolymers is preferably such that the resulting multiblock copolymer exhibits desired mechanical and thermal properties.

The content of prepolymer (A) in the multiblock copolymer of the invention can be 1-99% based on total weight of the multiblock copolymer, such as 5-95%, 10-90%, 20-80%, 30-70%, or 40-60%.

Prepolymer (A) may e.g. be prepared by ring-opening polymerisation. Thus, a prepolymer (A) may be a hydrolysable copolymer prepared by ring-opening polymerisation initiated by a diol or diacid compound, in one embodiment having a random monomer distribution. The diol compound can be an aliphatic diol or a low molecular weight polyether such as PEG. The prepolymer (A) synthesis by a ring-opening polymerisation is in one embodiment carried out in the presence of a catalyst. A suitable catalyst is Sn(Oct)₂ with M/I=5000-30 000 (M/I is the monomer to initiator ratio).

Prepolymer (A) may be a hydrolysable polyester, polyether ester, polycarbonate, polyester-carbonate, polyanhydride or copolymers thereof. The conditions for preparing such polymers are known in the art. For example, prepolymer (A) comprises reaction products of ester forming monomers selected from diols, dicarboxylic acids and hydroxycarboxylic acids.

The hydrolysable prepolymer (B) segment comprises the following structure:

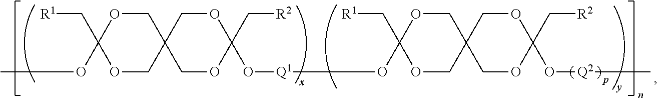

wherein
n is 4-100;
x is 0.25-1 and x+y=1;
p is 0 or 1;
R¹ and R² are independently selected from hydrogen and C₁-C₄ alkyl;
Q¹ is selected from

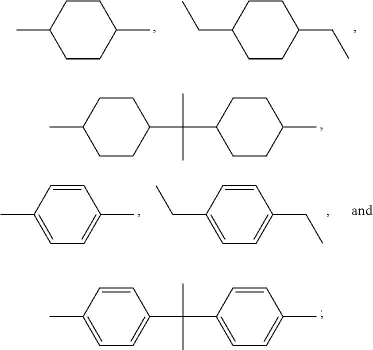

and
Q² is selected from

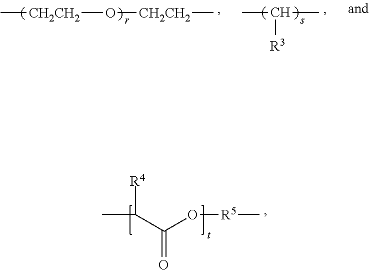

wherein
r is 1-100,
s is 1-12,
t is 1-10,
R³ is selected from hydrogen and C₁-C₆ alkyl,
R⁴ is selected from hydrogen and C₁-C₄ alkyl, and
R⁵ is selected from

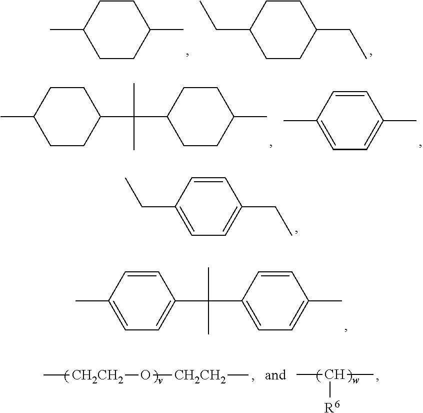

wherein
   v is 1-100,
   w is 1-12, and
   R⁶ is selected from hydrogen and C₁-C₆ alkyl.

Preferably, n is 5-50, such as 8-45, 10-40 or 12-35. Index x may be from 0.3-0.95, such as 0.4-0.9, or 0.5-0.8. In a preferred embodiment x is 1.

R¹ and R² are preferably independently selected from C₁-C₄ alkyl, such as methyl, ethyl, n-propyl, iso-propyl, n-butyl, iso-butyl, sec-butyl. More preferably R¹ and R² are both methyl.

Q¹ is preferably selected from

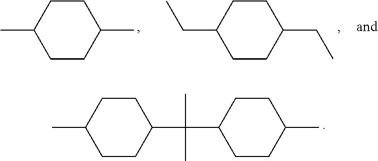

More preferably, Q is

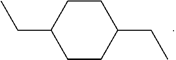

Q² is preferably selected from

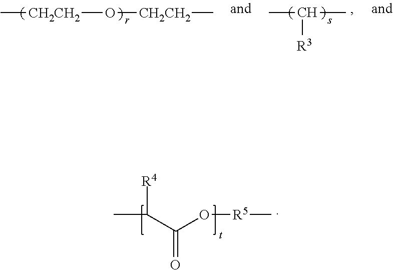

Preferably, r is 2-90, such as 5-80, or 10-70. Preferably, s is 2-10, such as 3-9, or 4-8. Preferably, t is 2-9, such as 3-8, or 4-7.

R³ is preferably selected from hydrogen and C₁-C₄ alkyl, such as methyl, ethyl, n-propyl, iso-propyl, n-butyl, iso-butyl, sec-butyl. More preferably, R³ is hydrogen or methyl.

R⁴ is preferably selected from hydrogen and methyl.

R⁵ is preferably selected from

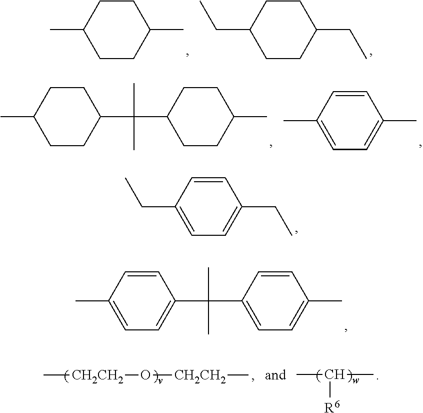

Preferably, u is 2-90, such as 5-80, or 10-70. Preferably, w is 2-10, such as 3-9, or 4-8.

R⁶ is preferably selected from hydrogen and C₁-C₄ alkyl, such as methyl, ethyl, n-propyl, iso-propyl, n-butyl, iso-butyl, sec-butyl. More preferably, R⁶ is hydrogen or methyl.

Preferably, the hydrolysable prepolymer (B) segment comprises, or consists of, the following structure.

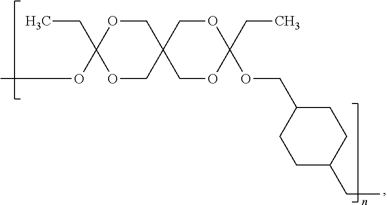

wherein n is 4-100, preferably 5-50, such as 8-45, 10-40 or 12-35.

Optional further monomers that may be present in the prepolymer (B) segment can be selected from lactic acid, glycolic acid and combinations thereof.

The hydrolysable prepolymer (B) segment may have a glass transition temperature T_{g} of 40° C. or more, preferably 50° C. or more, such as in the range of 60-100° C. The T_{g} can be determined by modulated differential scanning calorimetry (mDSC), as described in Example 2.

Prepolymer (B) can have a number average molecular weight (Mₙ) of 1000 g/mol or more, such as 2000 g/mol or more, 2500 g/mol or more, or 3000 g/mol or more. Prepolymer (B) can have a Mₙ of 10 000 g/mol or less, such as 9000 g/mol or less, or 8000 g/mol or less. The length of the prepolymers is preferably such that the resulting multiblock copolymer exhibits desired mechanical and thermal properties.

The content of prepolymer (B) in the multiblock copolymer of the invention can be 1-99% based on total weight of the multiblock copolymer, such as 5-95%, 10-90%, 20-80%, 30-70%, or 40-60%.

The prepolymer (B) can be synthesised by a polyaddition reaction between a diol and an acetal, more specifically from cyclohexane dimethanol (CHDM) and 3,9-divinyl-2,4,8,10-tetraoxaspiro[5.5]undecane (DVTOSU) to obtain a CHDM based poly(ortho ester) prepolymer (B). To obtain a bifunctional diol-functionalised poly(ortho ester), an excess of CHDM over DVTOSU should preferably be used and the polyaddition reaction can be monitored using NMR spectroscopy. Since poly(ortho ester) based polymers are known to hydrolyse rapidly under acidic conditions, a mild base amine, such as triethyl amine, may be added to the reaction medium to prevent hydrolysis and decrease of the molecular weight of the poly(ortho ester).

Optionally, an additional prepolymer segment (in addition to prepolymer segments (A) and (B)) may be present that is derived from a water-soluble polymer. This water-soluble polymer may be selected from the group consisting of one or more polyethers (such as polyethylene glycol (PEG), polytetramethyleneoxide (PTMO), polypropyleneglycol (PPG), and polytetramethylene ether glycol (PTMG)), or one or more other water-soluble polymers (such polyvinylalcohol (PVA), polyvinylpyrrolidone (PVP), polyvinylcaprolactam, poly(hydroxyethylmethacrylate) (poly(HEMA)), or polyphosphazenes). The additional prepolymer segment may also comprise copolymers of two or more of these polymers. For example, this additional water-soluble polymeric segment can be derived from PEG having a Mₙ of 150-5000 g/mol. The additional prepolymer segment that is derived from a water-soluble polymer can suitably be present in the multiblock copolymer in an amount of 60% or less by total weight of the multiblock copolymer, such as 50% or less, 40% or less, 30% or less, 20% or less, 10% or less, or 5% or less. The amount of the additional water-soluble polymer segment can be 0.1% or more by total weight of the multiblock copolymer, such as 1% or more, or 2% or more, 3% or more, 4% or more, or 5% or more.

The prepolymers will in one embodiment be linear and random (co)polyesters, polyester-carbonates, polyether esters, or polyanhydrides with reactive end-groups. These end-groups may be hydroxyl or carboxyl. It is preferred to have a dihydroxy terminated copolymer, but hydroxy-carboxyl or dicarboxyl terminated polymers can also be used. In case the polymer has to be linear, it can be prepared with a difunctional component (diol) as a starter, but in case a three or higher functional polyol is used, branched polyesters may be obtained.

The prepolymer segments of multiblock copolymer are linked by a multifunctional chain extender. This multifunctional chain extender is preferably a difunctional aliphatic chain extender. More preferably, the chain extender is a difunctional a diisocyanate, such as 1,4-butane diisocyanate. Nonetheless, it is also possible to use a trifunctional (or higher functional) chain extender, such as a tri-isocyanate. At sufficiently low conversion, this will result in a branched multiblock copolymer. Branched copolymers may show improved creep characteristics. It is also possible to obtain branched multiblock copolymers by using a difunctional chain extender, when at least one of the prepolymers has more than two functional groups.

The number of prepolymer blocks in the multiblock copolymers of the invention is preferably in the range of 2-1000, preferably 3-1000, such as 5-900, 10-800, 20-700, 30-600, or 40-500. This number of prepolymer blocks is preferably combined with a typical prepolymer block length in the range of 500 to 10 000 g/mol, such as 1000 to 7500 g/mol, more preferably 1200 to 5000 g/mol.

The multiblock copolymers of the invention may have an intrinsic viscosity of 0.1 dl/g or more, preferably 0.1-3 dl/g, more preferably 0.2-2 dl/g, such as 0.3-1 dl/g. Intrinsic viscosity can, for instance, be measured at 25° C. in chloroform via a single point method using an Ubbelohde Viscosimeter (DIN), type OC. These intrinsic viscosities approximately correspond to number average molecular weights (Mₙ) of 10 000 g/mol or more, preferably from 10 000 g/mol to 300 000 g/mol, more preferably from 20 000 g/mol to 200 000 g/mol, such as from 30 000 g/mol to 100 000 g/mol.

Preferably, the multiblock copolymers of the invention have a random distribution of the individual blocks in combination with a number average molecular weight in the range of from 10 000 g/mol to 300 000 g/mol and are solid under ambient conditions and under physiological conditions.

In a further aspect, the invention is directed to a process for preparing a biodegradable, thermoplastic multiblock copolymer according to the invention, comprising a chain-extension reaction of prepolymer (A) and prepolymer (B) in the presence of a multifunctional chain extender. This involves chain extension of prepolymer blocks with the multifunctional chain extender. Such a process results in a multiblock copolymer wherein the prepolymers are randomly distributed throughout the multiblock copolymer. Such copolymers cannot be obtained by synthesis processes that employ an addition reaction. In the multiblock copolymers of the invention, for example, the PEG may be randomly distributed throughout the polymer chain.

In this process, prepolymer (A), prepolymer (B) and the multifunctional chain extender may be as described herein.

Segmented multiblock copolymers can be made by chain-extending a mixture of prepolymers, in the desired ratio, with an equivalent amount of a multifunctional chain extender, in one embodiment an aliphatic molecule, such as 1,4-butanediisocyanate (BDI) or another diisocyanate. The segmented copolymers may be made in solution. Suitably, the prepolymer(s) are dissolved in an inert organic solvent and the chain extender is added pure or in solution.

The low polymerisation temperature and short polymerisation time will prevent transesterification and the monomer distribution is the same as in the prepolymers that build the copolymer. On the contrary, longer reaction times may lead to transesterification reactions and to a more random (i.e. less blocky) monomer distribution.

The materials obtained by chain-extending in the bulk can also be produced in situ in an extruder.

The multiblock copolymers of the invention preferably exhibit at least one glass transition temperature T_{g} of 30° C. or more, preferably 40° C. or more, such as 40-100° C. The multiblock copolymers may have more than one T_{g}, such as two or more T_{g}s. In an embodiment, the multiblock copolymers have two T_{g}s wherein a lower T_{g} is in the range of from −60° C. to 50° C. and a higher T_{g} is in the range of from 40° C. to 100° C.

The multiblock segmented copolymers can be formed into formulations of various shape and dimensions using any known technique such as, for example, solvent extraction/evaporation-based emulsification processes, extrusion, moulding, solvent casting, spray-drying, spray-freeze drying, electrospinning, or freeze drying. The latter technique is used to form porous materials. Porosity can be tuned by addition of co-solvents, non-solvents and/or leachables. Copolymers can be processed (either solid or porous) into microspheres, microparticles, nanospheres, rods, films, sheets, sprays, tubes, membranes, meshes, fibres, plugs, coatings and other articles. Products can be either solid, hollow or (micro)porous. A wide range of biomedical implants can be manufactured for applications in for example wound care, skin recovery, nerve regeneration, vascular prostheses, drug delivery, meniscus reconstruction, tissue engineering, coating of surgical devices, ligament and tendon regeneration, dental and orthopaedic repair. The copolymers can be used alone or can be blended and/or co-extruded with other absorbable or non-absorbable polymers.

In particular, the biodegradable multiblock copolymers of the invention are suitable as delivery vehicle for a polypeptide, allowing for the controlled release of the polypeptide from the matrix into its environment, e.g. in the body of a subject.

In yet a further aspect, the invention is directed to a composition for the delivery of at least one biologically active compound (e.g. a biologically active small molecule, protein or peptide) to a host, comprising the at least one biologically active compound encapsulated in a matrix, wherein said matrix comprises at least one biodegradable, thermoplastic multiblock copolymer as defined herein.

The composition may be in the form of one or more selected from the group consisting of microspheres, microparticles, nanoparticles, nanospheres, rods, solid implants, gels, in situ forming implants, coatings, films, sheets, sprays, tubes, membranes, meshes, fibres, plugs, and other configurations.

For example, the composition may be in the form of microspheres and/or microparticles. The average diameter of the microspheres and/or microparticles is then preferably in the range of 0.1-1000 μm, more preferably in the range of 1-100 μm, even more preferably in the range of 10-70 μm.

The composition may also be in the form of an in situ forming implant, wherein the biologically active compound is dissolved or suspended in a solution of the biodegradable, thermoplastic multiblock copolymer in an acceptable organic solvent such as n-methyl pyrrolidone (NMP), dimethyl sulphoxide (DMSO), benzyl benzoate (BB), benzyl alcohol, triacetin, glycofurol, low molecular weight polyethylene glycol. Following administration into the body, the solution may form in situ a depot by replacement of the organic solvent by aqueous body fluids, thereby entrapping the biologically active compound in the biodegradable, thermoplastic multiblock copolymer depot. Subsequently, the biologically active compound can be gradually released from the biodegradable, thermoplastic multiblock copolymer depot.

The composition may also be in the form of a solid implant which can, for instance, be prepared by hot-melt extrusion or injection moulding. The biologically active compound can be incorporated in the biodegradable, thermoplastic multiblock copolymer as a molecular blend or as a dispersion of solid particles.

The at least one biologically active compound in the composition preferably comprises a non-peptide, non-protein, small sized drug, and/or a biologically active polypeptide.

The multiblock copolymers of the invention have many options for tuning the release properties of the delivery composition for the specific application. The release rate of the biologically active compound may for example be increased by:
increasing the molecular weight of the water-soluble polymer in prepolymer (A) at constant molecular weight of prepolymer (A);
increasing the molar ratio between prepolymer (A) and prepolymer (B);
increasing the content of a monomer that gives a faster degrading polymer in prepolymer (A), e.g. by replacing ε-caprolactone by D,L-lactide or glycolide or by replacing D,L-lactide with glycolide;
decreasing the molecular weight of prepolymer (A) at a constant molecular weight of the water-soluble polymer and molar ratio between prepolymer (A) and prepolymer (B); and/or
using of an additional, third segment derived from a water-soluble polymer, whereby the content of the water-soluble polymer is increased.

The release rate may be decreased by the opposite changes as mentioned above.

Biologically active compounds which may be contained in the multiblock copolymer matrix include but are not limited to non-peptide, non-protein, small sized drugs having a molecular weight which in general is 1000 Da or less and biologically active polypeptides.

The at least one small-sized drug molecule may be present in the matrix in an amount of 0.1-80% by total combined weight of the matrix and the at least one small-sized drug molecule, in one embodiment 1.0-40%, and in another embodiment 5-20%. If it is desired to increase the hydrophilicity of the multiblock copolymer, and thereby increase the degradation rate of the copolymer and the release rate of the incorporated biologically active compound, the copolymer may be modified by replacing partially or completely the D,L-lactide of the hydrophilic prepolymer (A) segment by glycolide and/or by using a PEG component with a higher molecular weight or by increasing the weight fraction of PEG component in the prepolymer (A) segment. If it is desired to decrease the hydrophilicity of the polymer, and thereby decrease the degradation rate of the copolymer, and the release rate of the incorporated biologically active compound, the copolymer may be modified by replacing partially or completely the D,L-lactide of the hydrophilic prepolymer (A) segment by ε-caprolactone and/or by using a PEG component with a lower molecular weight or by decreasing the weight fraction of PEG component in the prepolymer (A) segment.

A polypeptide consists of amino acids linked by peptide bonds. Short polypeptides are also referred to as peptides, whereas longer polypeptides are typically referred to as proteins. One convention is that those polypeptide chains that are short enough to be made synthetically from the constituent amino acids are called peptides rather than proteins. However, with the advent of better synthetic techniques, polypeptides as long as hundreds of amino acids can be made, including full proteins like ubiquitin. Another convention places an informal dividing line at approximately 50 amino acids in length. This definition is somewhat arbitrary. Long polypeptides, such as the amyloid beta peptide linked to Alzheimer's disease, can be considered proteins; and small proteins, such as insulin, can be considered peptides. At any rate, the skilled person will appreciate that essentially any type of polypeptide can be encapsulated and subsequently released from a copolymer matrix.

In one embodiment, a composition of the invention comprises a biologically active peptide or biologically active protein.

The size of the polypeptide(s) can vary. In one embodiment, the polypeptide has a molecular weight of 10 000 Da or less. Polypeptides of such size are particularly suitable to be encapsulated in the matrix of a copolymer comprising PEG as a segment of prepolymer (A) and/or as an additional prepolymer, said PEG having a number average molecular weight of 400-3000 g/mol, or in another embodiment 600-1500 g/mol. Alternatively, or in addition, said PEG can be present in an amount of 5-60% by total weight of the copolymer, or in another embodiment 5-40%.

In another embodiment, said polypeptide is a biologically active protein having a molecular weight of 10 000 Da or more. These larger polypeptides are in one embodiment encapsulated in the matrix of a copolymer which contains PEG, as a segment of prepolymer (A) and/or as an additional prepolymer, and wherein said PEG has a number average molecular weight of 600-5000 g/mol, or in another embodiment 1000-3000 g/mol. Alternatively, or in addition, said PEG can be present in an amount of 5-70% by total weight of the copolymer, or in amount of 10-50%.

A composition of the invention can have any desirable appearance or shape. In one embodiment, multiblock copolymers of the current invention are processed in the form of microspheres, microparticles, sprays, an implant, a coating, a gel, a film, foil, sheet, membrane or rod.

One specific aspect relates to a composition in the form of microspheres. In general microspheres are fine spherical particles having a diameter of less than 1000 μm, and containing a biologically active compound. The microsphere may be a homogeneous or monolithic microsphere in which the biologically active compound is dissolved or dispersed throughout the polymer matrix. It is also possible that the microsphere is of a reservoir type in which the biologically active compound is surrounded by a polymer in the mononuclear or polynuclear state. When the biologically active compound is a small sized water-soluble drug, the drug may first be dispersed in a hydrophobic or lipophilic excipient, which combination then is dispersed in the form of particles, droplets, or micro-suspensions in the polymer matrix. Microspheres can then be formed from the emulsion.

The microspheres may be prepared by techniques known to those skilled in the art, including but not limited to coacervation, solvent extraction/evaporation, spray drying or spray-freeze drying techniques.

In one embodiment, the microspheres are prepared by a solvent extraction/evaporation technique which comprises dissolving the multiblock copolymer in an organic solvent such as dichloromethane, and emulsification of the multiblock copolymer solution in an aqueous phase containing an emulsifying agent, such as polyvinyl alcohol (as described among others by Okada, Adv. Drug Deliver. Rev. 1997, 28(1), 43-70).

The characteristics, such as particle size, porosity and drug loading of the so formed microspheres depend on the process parameters, such as viscosity or concentration of the aqueous polyvinyl alcohol phase, concentration of the multiblock copolymer solution, ratio of dichloromethane to aqueous solution of active, ratio of primary emulsion to polyvinyl alcohol phase and the stirring rate.

When the microspheres are formed by a spray-drying process, a low concentration of multiblock copolymer from 0.5-5% by total weight of the solution, in one embodiment about 2%, in the organic solvent, such as dichloromethane, is employed. Spray-drying results in general in the formation of porous, irregularly shaped particles.

As the microspheres are being formed, a biologically active compound is encapsulated in the microspheres or microparticles. In general, when the solvent extraction/evaporation technique is employed to encapsulate lipophilic compounds, the compound is first dissolved in the solution of the multiblock copolymer in an organic solvent such as dichloromethane or ethyl acetate. The organic solution is then subsequently emulsified in an aqueous polyvinyl alcohol solution, which yields an oil-in-water (O/W) emulsion. The organic solvent is then extracted into the aqueous phase and evaporated to solidify the microspheres.

In general, when the solvent evaporation technique is employed to encapsulate water-soluble compound, an aqueous solution of the compound is first emulsified in a solution of the multiblock copolymer in an organic solvent such as dichloromethane. This primary emulsion is then subsequently emulsified in an aqueous polyvinyl alcohol solution, which yields a water-in-oil-in-water (W/O/W) emulsion. The organic solvent, such as dichloromethane or ethyl acetate, is then extracted similarly to the O/W process route to solidify the microspheres. Alternatively, water-soluble agents may be dispersed directly in a solution of the multiblock copolymer in an organic solvent. The obtained dispersion is then subsequently emulsified in an aqueous solution comprising a surfactant such as polyvinyl alcohol, which yields a solid-in-oil-in-water (S/O/W) emulsion. The organic solvent is then extracted similarly to the O/W process route to solidify the microspheres.

When W/O/W and S/O/W emulsification routes are used to encapsulate water soluble compound, it may be challenging to obtain microspheres with sufficient encapsulation efficiency. Due to the water soluble character of the compound, part of the compound may be lost to the aqueous extraction medium such as aqueous polyvinyl alcohol solution. A viscosifier, such as gelatine, may be used in the internal water phase, to decrease diffusion of the compound in the internal water phase to the external water phase. Also, additives may be added to the external water phase to decrease the solubility of the compound in the external water phase. For this purpose, salts may be used or the pH may be adjusted.

Water-in-oil-in-oil (W//O) or solid-in-oil-in-oil (S/O/O) emulsification routes provide an interesting alternative to obtain microspheres with sufficient encapsulation efficiency. In the W/O/O process the biologically active compound is, similar to a W/O/W process, dissolved in an aqueous solution and emulsified with a solution of the polymer in an organic solvent, such as typically dichloromethane or ethyl acetate. Subsequently, a polymer precipitant, such as silicon oil, is then slowly added under stirring to form embryonic microparticles, which are then poured into heptane or hexane to extract the silicone oil and organic solvent and solidify the microspheres. The microparticles may be collected by vacuum filtration, rinsed with additional solvent and dried under vacuum. In the S/O/O emulsification route the biologically active compound is, similar to a S/O/W process, dispersed as a solid powder in a solution of the polymer in an organic solvent, such as dichloromethane or ethyl acetate. Subsequently, a polymer precipitant, such as silicon oil, is then slowly added under stirring to form embryonic microparticles, which are then poured into heptane or hexane to extract the silicone oil and dichloromethane and solidify the microspheres.

Stabilising agents may be added to the aqueous solution of protein to prevent loss of protein activity during processing into microspheres. Examples of such stabilising agents are polyvinyl alcohol (PVA), Tween®/polysorbatum, human serum albumin, gelatine and carbohydrates, such as trehalose, inulin and sucrose.

When the spray-drying technique is employed, an aqueous solution of the compound is emulsified in a solution of the copolymer in an organic solvent such as methylene chloride, as hereinabove described. The water-in-oil emulsion is then spray-dried using a spray dryer.

In further embodiments, the composition of the invention is in the form of a coating, an injectable gel, an implant (such as an injectable implant) or a coated implant. The composition in the form of a coating may be applied as a drug-eluting coating e.g. on a medical implant, such as a vascular or urinary stent, an orthopaedic prosthesis or an ocular implant.

Biologically active compounds may be formulated into injectable solid implants via hot melt extrusion. Typically, the compound and multiblock copolymer powders are physically mixed where after the resulting powder blend is introduced to the extruder, heated and processed to yield formulations of the desired shape and dimensions, such as a small diameter cylindrical rod. Instead of physical mixing of the compound and multiblock copolymer powders, the compound and polymer may be co-dissolved in a suitable solvent or a dispersion of compound in a solution of polymer in a suitable solvent may be prepared, followed by freeze-drying and extrusion of the freeze-dried powder. The latter generally improves the blend homogeneity and the content uniformity of the implants.

In yet another aspect the invention is directed to a method of delivering a biologically active compound to a subject in need thereof, comprising administering an effective dose of a composition as defined herein to said subject.

The subject is typically a mammal, preferably a human. However, veterinary use of the invention is also encompassed. The method can have a therapeutic, prophylactic, and/or cosmetic purpose. Any suitable mode of administration can be selected, depending on the circumstances. For example, administering may comprise the parenteral, oral, intra-arterial, intra-articular, intra-venal, intra-ocular, epidural, intra thecal, intra muscular, intra-peritoneal, intravenous, intra-vaginal, rectal, topical or subcutaneous administration of the composition. In one embodiment, the invention provides a method for delivering a biologically active polypeptide of interest to a subject in need thereof, comprising administering an effective dose of a composition according to the invention to said subject, wherein the composition is in the form of microspheres, an injectable implant or an in situ forming gel and wherein the composition is administered intraocularly, intra-arterially, intra muscularly or subcutaneously.

For topical administration, the microspheres may be contained in a gel, cream, or ointment, and may, if desired, be covered by a barrier. Thus, the microspheres may contain one or more biologically active compounds employed in the treatment of skin diseases, such as psoriasis, eczema, seborrhoea, and dermatitis.

In another embodiment, the microspheres may be contained in a gel such as a hyaluronic acid gel or a macromolecular polysaccharide gel. Such an embodiment is applicable particularly to parenteral applications, such as during and after surgery.

When administered via injection, the microspheres may be contained in a pharmaceutical carrier such as water, saline solution (for example, 0.9%), or a solution containing a surfactant in an amount of from 0.1-0.5% w/v. Examples of surfactants which may be employed include, but are not limited to, Tween 80 surfactant. The pharmaceutical carrier may further contain a viscosifier, such as sodium carboxymethylcellulose.

Such microspheres, when administered in combination with an acceptable pharmaceutical carrier, may be employed in the treatment of a variety of diseases or disorders, depending upon the biologically active compound that is encapsulated.

In one aspect, provided herein are injectable delivery systems comprising a multiblock copolymer as described herein.

The multiblock copolymer can be in the form of an implant. Such implant may be a microsphere, a rod, a film, a multiblock copolymer depot, or a plurality thereof. The multiblock copolymer may in the form of a plurality of polymeric microspheres that are each not less than 20 μm in diameter, wherein the polymeric microspheres comprise the multiblock copolymer as described herein. The polymeric microspheres can be from 20 μm to 80 μm in diameter, such as from 30 μm to 70 μm in diameter. The polymeric microspheres may be monodisperse with a coefficient of variation of about 25%. The injectable delivery systems may further comprise a therapeutic agent, or a pharmaceutically acceptable salt thereof. The therapeutic agent may be a small chemical, a protein, an antibody, a peptide or an oligonucleotide, or a combination thereof. Additionally, the injectable delivery systems can further comprise a pharmaceutically acceptable excipient.

The invention further relates to a medical device comprising a biodegradable, thermoplastic multiblock copolymer of the invention. Such a medical device can be in the form of microspheres, microparticles, nanoparticles, nanospheres, rods, solid implants, gels, in situ forming implants, coatings, films, sheets, sprays, tubes, membranes, meshes, fibres, scaffolds or plugs. Preferably, the medical device further comprises at least one biologically active compound encapsulated in the matrix of the biodegradable, thermoplastic multiblock copolymer, and which biologically active compound can be controllably released after insertion in a human or animal.

The invention has been described by reference to various embodiments, compositions and methods. The skilled person understands that features of various embodiments, compositions and methods can be combined with each other.

All references cited herein are hereby completely incorporated by reference to the same extent as if each reference were individually and specifically indicated to be incorporated by reference and were set forth in its entirety herein.

The use of the terms “a” and “an” and “the” and similar referents in the context of describing the invention (especially in the context of the claims) are to be construed to cover both the singular and the plural, unless otherwise indicated herein or clearly contradicted by context. The terms “comprising”, “having”, “including” and “containing” are to be construed as open ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. For the purpose of the description and of the appended claims, except where otherwise indicated, all numbers expressing amounts, quantities, percentages, and so forth, are to be understood as being modified in all instances by the term “about”. Also, all ranges include any combination of the maximum and minimum points disclosed and include any intermediate ranges therein, which may or may not be specifically enumerated herein.

For the purpose of clarity and a concise description features are described herein as part of the same or separate embodiments, however, it will be appreciated that the scope of the invention may include embodiments having combinations of all or some of the features described.

### EXAMPLES

The invention will now be further illustrated by the following non-limiting examples.

In the following examples various biodegradable thermoplastic amorphous poly(ortho ester) based multiblock copolymers were synthesised and evaluated for their processability into long-acting injectable drug delivery formulations, their drug release characteristics and their erosion characteristics. The polymers were composed of an amorphous poly(ortho ester)-based prepolymer (B) segment with a high T_{g} and a prepolymer (A) segment comprising a polyester or a polyether ester polymer block.

### Example 1

PLGA polymers are most often used for sustained release of drugs and have been clinically proven to be safe in the body. Even though PLGA polymers are fairly versatile, and their physiochemical properties can be tuned to accommodate different drug delivery needs, their suitability has been shown to be limited in protein delivery. Protein stability remains a major obstacle in delivering proteins with PLGA due to (1) the hydrophobic character of the polymers, (2) the formation of acidic degradation products and the accumulation of acidic degradation products in the polymer matrix leading to an in situ pH drop due to which the any encapsulated proteins may degrade and lose their biological activity. Proteins have also been shown to be (3) chemically modified through deamination or acylation within the PLGA matrix. Consequently, delivery systems made with PLGA are associated with all the issues as mentioned above including (4) protein aggregation and (5) undesirable release kinetics.

PCL (poly(ε-Caprolactone)-PEG-poly(ε-Caprolactone)-based hydrophilic block in combination with a poly(L-lactide)-based crystalline block) multiblock copolymers composed of a crystalline poly(L-lactide) block with a number average molecular weight (Mₙ) of 4000 g/mol (abbreviated as LL40) in combination with a hydrophilic poly(ε-caprolactone)-PEG1000-poly(ε-caprolactone) block with Mₙ of 2000 g/mol and 50 wt. % of PEG with a molecular weight of 1000 g/mol (PEG1000) (abbreviated as CP10C₂₀) in block ratios varying from 20/80 (20CP10C20-LL40) to 50/50 (50CP10C20-LL40) and PCL multiblock copolymer composed of LL40 in combination with a hydrophilic poly(ε-caprolactone)-PEG3000-poly(ε-caprolactone) block with Mₙ of 4000 g/mol and 75 wt. % of PEG with a molecular weight of 3000 g/mol (PEG3000) (abbreviated as CP30C40) in a 30/70 (30CP30C40-LL40) or 50/50 ((50CP30C40-LL40) weight ratio were found suitable for sustained release delivery of biologics of different molecular size such as goserelin, lysozyme, bovine serum albumin, insulin-like growth factor-1 (WO-A-2012/005594), hepatocyte growth factor and vascular endothelial growth factor (Scheiner et al., J. Pharm Sci. 2020, 109, 863-870).

Unfortunately, 50CP10C20-LL40-based microspheres were found to degrade very slowly. Based on extrapolation of experimental data, the in vitro erosion time of the 50CP10C20-LL40 microspheres is projected to be 3-4 years (FIG. 1) and at least 14-16 months in vivo. The slow erosion of PCL multiblock copolymers was confirmed for other PCL multiblock copolymers such as 20CP10C20-LL40 and 30CP30C40-LL40 and attributed to slow hydrolysis of the crystalline poly(L-lactide) block. Furthermore, it was found that the melting enthalpy (and thus the degree of crystallisation) of multiblock copolymers containing a crystalline block was highly dependent on the processing conditions. Variation in the degree of crystallinity of the polymer matrix can potentially lead to (unacceptable) variation of critical product characteristics such as release kinetics and hence lead to reproducibility issues and unacceptable inter-batch variability. Furthermore, due to ongoing crystallisation of an incompletely crystallised polymer matrix during long-term product storage, the same critical product characteristics may change over time leading to drug product stability issues.

In accordance with the invention the crystalline poly(L-lactide) block is replaced by an amorphous poly(ortho ester) block with a high T_{g}. By careful selection of diols used in poly(ortho ester) polymer synthesis, a prepolymer segment (B) with a T_{g}>60° C. can be obtained. In case of phase separation of such a high T_{g} prepolymer segment (B) and a low T_{g} prepolymer segment (A), a multiblock copolymer with two T_{g}s will be obtained, one T_{g} representing the domains formed by the high T_{g} prepolymer segment (B) and one T_{g} representing the domains formed by the low T_{g} prepolymer segment (A). The mechanical characteristics of the multiblock copolymer will to a large extent be determined by the high T_{g} domains, similar as observed for semi-crystalline phase separated multiblock copolymers of WO-A-2012/005594 and WO-A-2013/015685. The high T_{g} domains can furthermore act as physical crosslinks thereby providing further control over the drug release kinetics. In case of phase mixing of the high T_{g} prepolymer segment (B) and a low T_{g} prepolymer segment (A), a multiblock copolymer with a single T_{g} with a value between the individual T_{g}s of the high T_{g} prepolymer segment (B) and the low T_{g} prepolymer segment (A) will be obtained. Hence the resulting T_{g} of the multiblock copolymer can be sufficiently high, such as around 40° C. or higher.

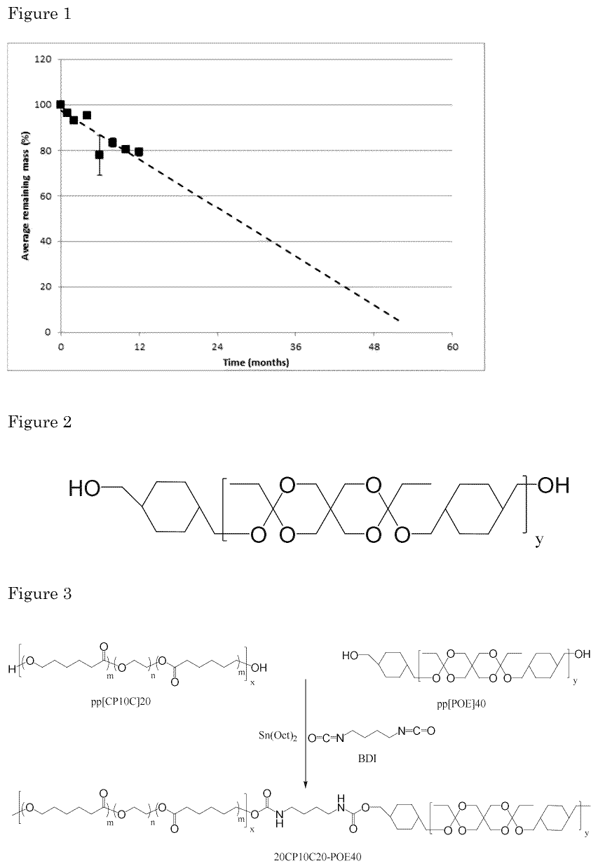

A high T_{g} poly(ortho ester) prepolymer block was synthesised by polyaddition reaction between cyclohexane dimethanol (CHDM) and 3,9-divinyl-2,4,8,10-tetraoxaspiro[5.5]undecane (DVTOSU) to obtain a CHDM based poly(ortho ester) prepolymer block, hereafter abbreviated as POE. To obtain a bifunctional POE block, up to 10 wt. % excess of CHDM over DVTOSU was used and the polyaddition reaction was monitored using NMR spectroscopy. The molecular weight of the POE prepolymer segments (B) was determined using GPC relative to polystyrene standards and NMR spectroscopy with an internal standard. The diol-functionalised CHDM based POE prepolymer (FIG. 2) had a Mₙ of 3.8-4.1 kg/mol (hereafter abbreviated as POE40) and a T_{g} of 65-69° C. Since POE based polymers are known to hydrolyse rapidly under acidic conditions, a mild base triethyl amine was added in the precipitation medium to prevent hydrolysis and the dried yellow powder was stored at <−10° C. in moisture free condition.

A poly(ε-caprolactone)-co-PEG1000-co-poly(ε-caprolactone) prepolymer with a target Mₙ of 2000 g/mol (abbreviated as ppCP10C20) was prepared by ring-opening polymerisation of ε-caprolactone using polyethylene glycol with a molecular weight of 1000 g/mol (PEG1000) as initiator. In brief, ε-caprolactone (Acros Organics) was dried and distilled over CaH₂ under reduced pressure and stannous octoate (Sigma Corp.) was purified by vacuum distillation. About 497.7 g (0.49 mol) of PEG1000, Merck, Emprove® Essential Ph Eur) was weighed into a three-necked bottle under nitrogen atmosphere and dried at 90° C. under reduced pressure for at least 16 h and then 503.3 g (4.34 mol) of distilled ε-caprolactone was added to the PEG under nitrogen atmosphere and the mixture was heated to 160° C. Subsequently, 101 mg of distilled stannous octoate (monomer/catalyst ratio of 17 600 mol/mol) was added and the mixture was magnetically stirred and heated at 160° C. until conversion was >98% as confirmed by ¹H-NMR. Molecular weight as determined by ¹H-NMR was ˜2000 g/mol. Similarly, ppCP30C40, a poly(ε-caprolactone)-co-PEG3000-co-poly(ε-caprolactone) prepolymer with a target Mₙ of 4000 g/mol was prepared by using PEG3000 as an initiator.

[Poly(ε-caprolactone)-co-PEG-co-poly(ε-caprolactone)]-b-[poly(ortho ester)] multiblock copolymers with block ratios of 20/80 w/w (hereafter abbreviated as 20CP10C20-POE40) (FIG. 3) and 50/50 (hereafter abbreviated as 50CP10C20-POE40) were synthesised by chain-extending the diol-functionalised POE40 with CP10C20 using 1,4-butanediisocyanate as a chain extender. In brief, both prepolymers were dried overnight under reduced pressure. After drying ppPOE40 in situ in a flange glass reactor, the desired amount of ppCP10C20 or ppCP30C40 prepolymer was added. Water-free distilled p-dioxane was added into the reactor until a polymer concentration of 30 wt. % was reached. The reactor was heated to 80° C. to dissolve the prepolymers and once a homogeneous solution was obtained, 1,4-butanediisocyanate (BDI) (Actu-All Chemicals) was added. Additional stannous octoate was added to increase its total content to 50-120 ppm and the reaction mixture was stirred mechanically until the desired viscosity was obtained, where after distilled p-dioxane containing 20 wt. % water. Stirring was continued for an additional 30 minutes. The reaction mixture was further diluted with p-dioxane to a polymer concentration of 10 wt. %, cooled to room temperature, poured into a tray, frozen at −20° C. after which p-dioxane was removed from the frozen solution under reduced pressure yielding dry polymer. Using similar procedures, a 50CP30C40-POE40 multiblock copolymer was prepared by chain-extending ppCP30C40 prepolymer with POE40 prepolymer in a 50/50 w/w ratio using BDI as a chain extender. Table 1 lists the experimental details of the various [poly(ε-caprolactone)-co-PEG-co-poly(ε-caprolactone)]-b-[poly(ortho ester)] multiblock copolymers.

Polymers were analysed for polymer composition (¹H-NMR), intrinsic viscosity and thermal properties (mDSC) as described in Example 2. Table 2 shows the results from characterisation of the multiblock copolymers prepared.

TABLE 1
Experimental details of [poly(ε-caprolactone)-
co-PEG-co-poly(ε-caprolactone)]-b-[poly(ortho
ester)] multiblock copolymers.

| | | | | ppCP10C20 or | | |
|---|---|---|---|---|---|---|
| | | ppPOE40 | | ppCP30C40 | | |
| | | | Mₙ | | Mₙ | BDI |
| Grade | RCP | (g) | (g/mol) | (g) | (g/mol) | (g) |
| 50CP10C20-POE40 | 1652 | 15.00 | 4400 | 14.83 | 2000 | 1.92 |
| 20CP10C20-POE40 | 1679 | 66.40 | 4400 | 16.73 | 2000 | 4.10 |
| 50CP30C40-POE40 | 1653 | 15.27 | 4400 | 15.35 | 4000 | 1.18 |

TABLE 2
Chemical composition, intrinsic viscosity and thermal
characteristics of multiblock copolymers

| | | Block ratio | | IV | T_{g} | Tₘ |
|---|---|---|---|---|---|---|
| Grade | RCP | Target | ¹H-NMR | (dl/g) | (° C.) | (° C.) |
| 50CP10C20- | 1636 * | 50/50 | 46.9/53.1 | 1.08 | −51 | 129 |
| LL40 | | | | | | |
| 50CP10C20- | 1652 | 50/50 | 56.7/43.3 | 0.92 | −53, 47 | |
| POE40 | | | | | | |
| 20CP10C20- | 1679 | 20/80 | 26.0/74.0 | 0.61 | −44, 51 | — |
| POE40 | | | | | | |
| 50CP30C40- | 1653 | 50/50 | 56.1/43.9 | 0.65 | −38, 73 | 41 ** |
| POE40 | | | | | | |

* RCP1636 (50CP10C20-LL40 is added as a reference)
** Tₘ of PEG

The [poly(ε-caprolactone)-co-PEG-co-poly(ε-caprolactone)]-b-[poly(ortho ester)] multiblock copolymers exhibited two glass transitions. The presence of two glass transitions is due to microphase separation of incompatible low T_{g} poly(ε-caprolactone)-co-PEG-co-poly(ε-caprolactone)] based domains and high T_{g} poly(ortho ester) based domains. In this way, the poly(ortho ester) prepolymer segments (B) provide good structural integrity to the multiblock copolymer even when combined with low T_{g} prepolymer segment (A).

The synthesised poly(ortho ester) multiblock copolymers (POE-MBCPs) were evaluated for their processability (particle size distribution, microscopic appearance, stickiness, absence of agglomeration) into polymer-only microspheres. Polymers that were well processable into microspheres were further evaluated for their in vitro erosion kinetics.

Polymer-only microspheres were prepared by a solvent extraction/evaporation based oil-in-water emulsification process. About 5.8 g of polymer dissolved in 52.4 g of dichloromethane (10.0 wt. %) was emulsified in 3.08 kg of ultrapure water containing 4.0 wt. % polyvinylalcohol (PVA) and 5 wt. % NaCl via membrane emulsification using a membrane with a pore size of 20 μm. The resulting microspheres were collected on a 5 μm membrane filter and washed three times with 250 ml of ultrapure water containing 0.05 wt. % of Tween® 80 and three times with 250 g of ultrapure water. Finally, the microspheres were lyophilised.

The particle size distribution, including average diameter and coefficient of variance, of the polymer-only microspheres was measured by laser diffraction (Horiba® LA-960 Laser Particle Size Analyser). Microspheres were suspended in water until transmittance was within 70-90% and the particle size distribution of the suspension was determined within the range of 10 nm-5000 μm. The surface morphology of the microspheres was evaluated by scanning electron microscopy, using a JEOL JCM-5000 Neoscope. A small amount of microspheres was adhered to carbon conductive tape and coated with a gold layer. The sample was imaged using a 10 kV electron beam.

The in vitro erosion of polymer-only microspheres was measured in 100 mM of phosphate buffer pH 7.4 (90-100 mg of microspheres in 10 ml). The samples were incubated at 37° C. At each sampling point, the microspheres were collected, freeze-dried and weighed.

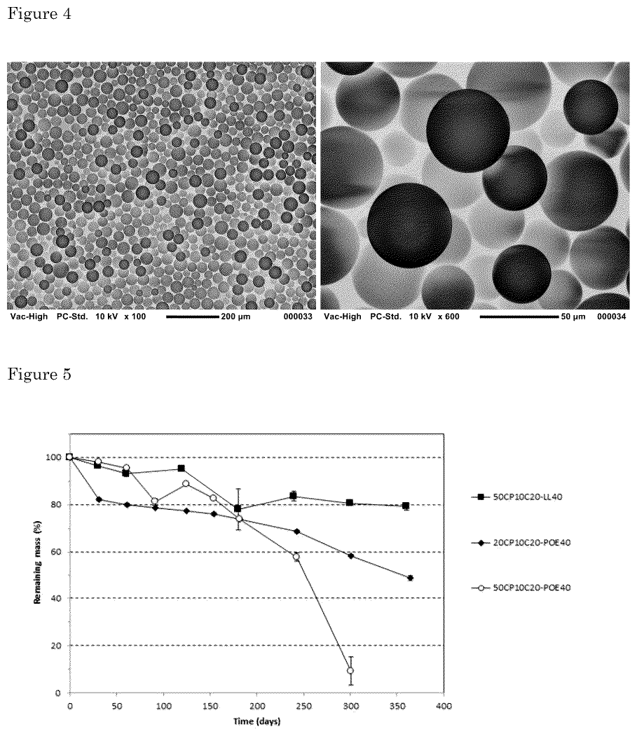

The POE-MBCPs composed of a POE40 based prepolymer (B) segment in combination with a hydrophilic poly(ε-caprolactone)-PEG-poly(ε-caprolactone) prepolymer (A) segment were well processable allowing the manufacturing of spherical microspheres with a smooth surface as shown in the SEM images in FIG. 4. The 20CP10C20-POE40 based microspheres had a volume averaged particle size (D₅₀(vol)) of 60.9 μm, whereas the 50CP10C20-POE40 based microspheres were slightly larger with D₅₀(vol) of 64.5 μm.

The in vitro erosion kinetics of the polymer-only POE-MBCPs based microspheres is shown in FIG. 5. The POE-MBCP based microspheres were found to erode significantly faster in vitro as compared to the PCL multiblock copolymer composed of a crystalline poly(L-lactide) prepolymer (B) segment in combination with a hydrophilic poly(ε-caprolactone)-PEG-poly(ε-caprolactone) prepolymer (A) segment. By replacing the poly(L-lactide) prepolymer (B) segment of 50CPC10C20-LL40 by a poly(ortho ester) prepolymer (B) segment, the resulting 50CP10C20-POE40 was found to erode completely in around 300 days.

Sustained release microspheres were prepared of ICP002, a recombinant protein with a molecular weight of ˜35 kDa, using 20CP10C20-POE40, 50CP10C20-POE40 and blends thereof. Microspheres with a target ICP002 protein loading of approximately 4 wt. % were prepared by solvent extraction/evaporation using a W1/O/W2 water-in-oil-in-water double emulsion-based membrane emulsification process. About 1 to 2 g of polymer was dissolved in dichloromethane (0) to a concentration of 15 wt. % and filtered over a 0.2 μm PTFE filter. Aqueous protein solution (W1) with a concentration of ˜100 mg/ml was added to a O/W1 ratio of 14-15 vol/vol followed by emulsification using a rotor-stator mixer (21 600 rpm, 40 seconds) to yield a primary emulsion. The primary emulsion was then emulsified with an aqueous solution (4.0 wt. % PVA, 5 wt. % of NaCl in ultrapure water) (W2) by membrane emulsification using a membrane with 20 μm pores and a CP/DP ratio of ˜50 vol/vol to form a secondary emulsion. The secondary emulsion was stirred for 4 hours at room temperature to remove dichloromethane by solvent extraction/evaporation. The resulting microspheres were collected on a 5 μm membrane filter and washed three times with aqueous 0.05 w/v % Tween® 80 solution and three times with ultrapure water, after which the hardened microspheres were dried by lyophilisation.

The particle size distribution, including average diameter and coefficient of variance, of the microspheres was measured with a Coulter Counter Multisizer III using a 200 or 400 μm aperture and 20 000 counts. The volume average particle size (D₅₀(vol)) and coefficient of variance (C.V.) were determined in the range of 4-200 μm. The ICP002-loaded microspheres had D₅₀(vol) varying between 50 and 80 μm and a narrow particle size distribution with a C.V. of 15-20%.

The surface morphology of the microspheres as evaluated by scanning electron microscopy showed that 50CP10C20-POE40-based ICP002 loaded microspheres had a rough surface morphology and showed extensive agglomeration. Upon increasing the weight fraction of 20CP10C20-POE40 in the 50CP10C20-POE40/20CP10C20-POE40 blends used to prepare the ICP002 loaded microspheres, the surface roughness of the microspheres decreased. ICP002 loaded microspheres prepared of 100% 20CP10C20-POE40 had a very smooth surface morphology. The ICP002 loading of the microspheres as indirectly determined from the maximum amount of ICP002 released in vitro varied between 1.2 and 2.2 wt. %, as shown in Table 3.

TABLE 3
Characteristics of protein ICP002 loaded microspheres prepared
of 20CP10C20-POE40, 50CP10C20-POE40 and blends thereof.

| | Polymer | Average particle size | ICP002 |
|---|---|---|---|
| | blend | (D₅₀(vol)) | content ** |
| Code | ratio * | (μm) | (wt. %) |
| PBD17-008 | 0/100 | 72 (CV 15%) | 2.2 |
| PBD17-027 | 33/67 | 78 (CV 18%) | 2.1 |
| PBD17-025 | 50/50 | 72 (CV 16%) | 1.7 |
| PBD17-024 | 67/33 | 70 (CV 17%) | 2.0 |
| PBD17-011 | 100/0 | 52 (CV 21%) | 1.2 |

* 20CP10C20-POE40/50CP10C20-POE40 polymer blend ratio (wt. %/wt. %)
** protein loading was determined from maximum recovery during in vitro release testing

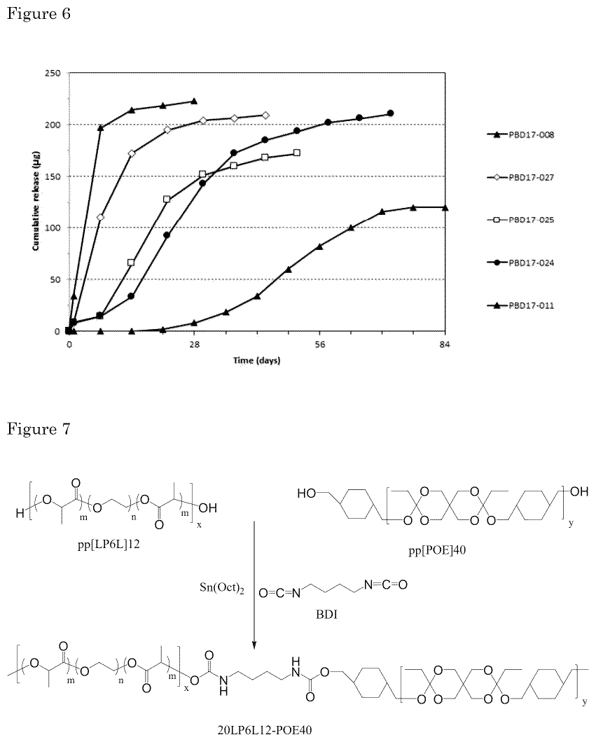

In vitro release (IVR) studies of ICP002 loaded microspheres were conducted in triplicate in 2 ml of 100 mM phosphate buffer pH 7.4 containing 0.02 w/v % NaN₃) thermostated at 37° C. Samples were taken at pre-determined time points and analysed with RP-UPLC to establish the cumulative protein release against sampling time. Most ICP002 loaded microspheres exhibited sigmoidal release kinetics (FIG. 6) with a lag time, release rate and release duration that were dependent on the 20CP10C20-POE40/50CP10C20-POE40 polymer blend ratio. The release rate decreased by increasing the weight fraction of 20CP10C20-POE40 in the polymer blend.

### Example 2

This example describes the analytical methods used for the characterisation of prepolymers and multiblock copolymers. ¹H-NMR was performed on a Bruker Avance DRX 500 MHz NMR spectrometer (B AV-500) equipped with Bruker Automatic Sample Changer (BACS 60) (Varian) operating at 500 MHz. The d₁ waiting time was set to 20 s, and the number of scans was 16. Spectra were recorded from 0 to 14 ppm. The conversion in prepolymers and the block ratio in MBCPs was determined from ¹H-NMR. The Mₙ of the prepolymer segments (A) was determined from both in weights and ¹H-NMR. ¹H-NMR samples were prepared by adding 1.3 g of deuterated chloroform to 25 mg of polymer.

Intrinsic viscosity of MBCPs was measured using an Ubbelohde Viscosimeter (DIN), type OC, Si Analytics supplied with a Si Analytics Viscosimeter including a water bath. The measurements were performed in chloroform at 25° C. The polymer concentration in chloroform was such that the relative viscosity was in the range of 0.28-2.0 dl/g.

Modulated differential scanning calorimetry (mDSC) was used to determine the thermal behaviour of the multiblock copolymers using a Q2000 MDSC (TA instruments, Ghent, Belgium). About 4-8 mg of dry material was accurately weighed and heated under a nitrogen atmosphere from −85° C. to 100° C. at a heating rate of 2° C./min and a modulation amplitude of +/−0.42° C. every 80 seconds. The glass transition temperature (T_{g}, midpoint) was determined from the reversing heat flow. Temperature and enthalpy were calibrated with an indium standard.

### Example 3

In this example, procedures for the preparation of prepolymer comprising poly(D,L-lactide)-co-PEG-co-poly(D,L-lactide), is provided. Poly(D,L-lactide)-co-PEG600-co-poly(D,L-lactide) prepolymer with a target Mₙ of 1200 g/mol (abbreviated as ppLP6L12) was prepared by ring-opening polymerisation of D,L-lactide using PEG with a molecular weight of 600 g/mol (PEG600) as initiator. 252.4 g (1.75 mol) of D,L-lactide (Purac) was weighed into a three-necked bottle under nitrogen atmosphere and dried at 50° C. for at least 16 h under reduced pressure. 249.5 g (0.42 mol) of pre-dried PEG600 (Merck, Emprove® Essential Ph Eur) was added under a nitrogen atmosphere. The mixture was heated to 140° C. 51 mg of stannous octoate was added and the mixture was magnetically stirred and reacted at 140° C. during 22 h. ¹H-NMR showed 96.0% monomer conversion. Molecular weight as determined by ¹H-NMR was 1201 g/mol.

### Example 4

In this example, procedures for the preparation of prepolymers comprising poly(D,L-lactide) and poly(D,L-lactide-co-glycolide) are provided. Poly(D,L-lactide) prepolymer with a target Mₙ of 4000 g/mol (abbreviated as ppL40) was synthesised in bulk by 1,4-butanediol (BDO) initiated ring-opening polymerisation. BDO (Acros Organics) was distilled over CaH₂ under reduced pressure and stored under nitrogen atmosphere until further use. 509.6 g (3.53 mol) of D,L-lactide (Purac) was weighed into a three-necked flask under nitrogen atmosphere and dried at 50° C. for at least 16 h under reduced pressure. Subsequently, 11.4 g (0.13 mol) BDO was added to the monomer under nitrogen atmosphere. The mixture was heated to 140° C. giving a clear molten fluid. 62 mg of stannous octoate was added as 1.0 wt % solution in p-dioxane (Acros, dried and distilled), starting the ring-opening polymerisation. After 20 h the reaction is cooled down to room temperature. ¹H-NMR showed 97.0% monomer conversion. Molecular weight as determined by ¹H-NMR was 4120 g/mol.

Poly(D,L-lactide-co-glycolide) prepolymer with a target Mₙ of 4000 g/mol (abbreviated as ppGL40) was prepared by ring-opening copolymerisation of D,L-lactide and glycolide using BDO as an initiator. 66.6 g (0.46 mol) of D,L-lactide (Purac) and 53.7 g (0.46 mol) of glycolide (Purac) were added into a three-necked flask under nitrogen and dried at 50° C. for at least 16 h under reduced pressure. When dried, 2.7 g (0.03 mol) of distilled BDO was added to the monomers under nitrogen atmosphere. The reaction mixture was heated to 130° C. Once the clear molten fluid was obtained, 14.1 mg of distilled stannous octoate was added as 1 wt. % solution in distilled p-dioxane. The reaction was stirred for an additional 27 h and cooled down when conversion had reached 97.2%. Molecular weight determined by ¹H-NMR was 4150 g/mol.

### Example 5

This example describes the synthesis and characterisation of [poly(D,L-lactide)-co-PEG600-co-poly(D,L-lactide)]-b-[polyorthoester] multiblock copolymer with a block ratio of 20/80 w/w (20LP6L12-POE40). FIG. 7 shows its synthesis and molecular structure. 20LP6L12-POE40 (RCP2131) was prepared by chain-extension of 64.0 g of ppPOE40 prepolymer with Mₙ of 4100 g/mol with 16.0 g of ppLP6L12 prepolymer with Mₙ of 1200 g/mol using 1.21 g BDI as a chain extender. After drying the prepolymers, they were added into a flange glass reactor in the required amount and dissolved in distilled p-dioxane at a concentration of 30 wt. % after which BDI was added. Chain extension, work-up and drying of 20LP6L12-POE40 was performed according to the procedures as described in Example 1.

The polymer was analysed for polymer composition (¹H-NMR), intrinsic viscosity and thermal properties (mDSC) as described above. The block ratio as determined from ¹H-NMR was 20.0/80.0 w/w. The polymer had an intrinsic viscosity (IV) of 0.28 dl/g and a T_{g} of 57° C.

### Example 6

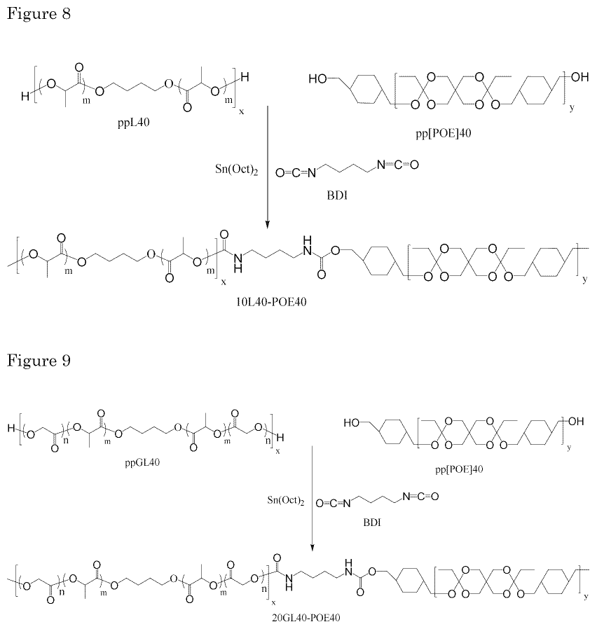

This example describes the synthesis and characterisation of [poly(D,L-lactide)]-b-[poly(ortho ester)] or [poly(D,L-lactide-co-glycolide)]-b-[poly(ortho ester)] multiblock copolymers (FIGS. 8 and 9). [Poly(D,L-lactide)]-b-[poly(ortho ester)] and [poly(D,L-lactide-co-glycolide)]-b-[poly(ortho ester)] multiblock copolymers with various block ratios were prepared by chain-extension of ppPOE40 prepolymer with ppL40 or ppGL40 prepolymers using BDI as a chain extender. Both prepolymers in desired amounts were dried directly in flange glass reactor overnight. Subsequently, distilled p-dioxane was added to obtain a clear polymer solution at a concentration of 30 wt. % after which BDI was added. Chain extension, work-up and drying of the [poly(D,L-lactide)]-b-[poly(ortho ester)] and [poly(D,L-lactide-co-glycolide)]-b-[poly(ortho ester)] multiblock copolymers was performed according to the procedures as described in Example 1. Table 4 depicts the experimental details of the various [poly(D,L-lactide)]-b-[poly(ortho ester)] and [poly(D,L-lactide-co-glycolide)]-b-[poly(ortho ester)] multiblock copolymers.

TABLE 4
Experimental details of [poly(D,L-lactide)]-b-[poly(ortho
ester)] and [poly(D,L-lactide-co-glycolide)]-b-[poly(ortho
ester)] multiblock copolymers.

| | | ppPOE40 | | ppL40 or ppGL40 | | |
|---|---|---|---|---|---|---|
| | | | Mₙ | | Mₙ | BDI |
| Grade | RCP | (g) | (g/mol) | (g) | (g/mol) | (g) |
| 100POE40 | 1647 | 30.12 | 4400 | — | — | 1.00 |
| 10L40-POE40 | 1650 | 32.66 | 4400 | 3.48 | 4040 | 1.34 |
| 25L40-POE40 | 1649 | 24.67 | 4400 | 8.18 | 4040 | 1.23 |
| 50L40-POE40 | 1648 | 14.92 | 4400 | 14.92 | 4040 | 1.14 |
| 25GL40-POE40 | 1654 | 29.64 | 4400 | 9.53 | 4120 | 1.51 |

Polymers were analysed for chemical composition (¹H-NMR), intrinsic viscosity and thermal properties (mDSC) as described above. Table shows the collected analysis results for prepared multiblock copolymers. As anticipated, the T_{g} of a multiblock copolymer solely consisting of POE40-based blocks (100POE40) was very high (90° C.). Upon introducing small amounts (10-25 wt. %) of a poly(D,L-lactide) block the T_{g} of the resulting multiblock copolymers reduced slightly to 75-85° C. By chain-extending the POE40 prepolymer block with a poly(D,L-lactide-co-glycolide) prepolymer block, T_{g} reduction of the resulting multiblock copolymers was higher.

TABLE 5
Collected results regarding the chemical composition, intrinsic
viscosity and residual dioxane content of multiblock copolymers

| | | Block ratio | | IV | T_{g} |
|---|---|---|---|---|---|
| Grade | RCP | Target | ¹H-NMR | (dl/g) | (° C.) |
| 100POE40 | 1647 | 100/0 | N.A. | 0.32 | 90 |
| 10L40-POE40 | 1650 | 10/90 | 10.2/89.9 | 0.33 | 85 |
| 25L40-POE40 | 1649 | 25/75 | 29.0/71.0 | 0.35 | 75 |
| 50L40-POE40 | 1648 | 50/50 | 53.9/46.1 | 0.48 | 50 |
| 25GL40-POE40 | 1654 | 25/75 | 27.8/72.2 | 0.28 | 48 |

### Example 7

To study the erosion kinetics of the POE-based multiblock copolymers, polymer-only microspheres were prepared by solvent extraction/evaporation based oil-in-water emulsification. 5.8 g of polymer dissolved in 52.4 g of dichloromethane (10.0 wt. %) was emulsified in 3.08 kg of ultrapure water containing 4.0 wt. % PVA and 5 wt. % NaCl via membrane emulsification using a membrane with a pore size of 20 μm. The resulting microspheres were collected on a 5 μm membrane filter and washed three times with 250 ml of ultrapure water containing 0.05 wt. % of Tween® 80 and three times with 250 g of ultrapure water. Finally, the microspheres were lyophilised.

The particle size distribution of the microspheres was measured by laser diffraction (Horiba® LA-960 Laser Particle Size Analyser). Microspheres were suspended in water until transmittance was within 70-90% and the particle size distribution of the suspension was determined within the range of 10 nm-5000 μm. The surface morphology of the microspheres was evaluated by scanning electron microscopy, using a JEOL JCM-5000 Neoscope. A small amount of microspheres was adhered to carbon conductive tape and coated with gold for 3 min. The sample was imaged using a 10 kV electron beam.

The in vitro erosion of non-loaded polymer-only microspheres was measured in 100 mM of phosphate buffer pH 7.4 (90-100 mg of microspheres in 10 ml). The samples were incubated at 37° C. At each sampling point, the microspheres were collected, freeze-dried and weighed.

All POE-based multiblock copolymers were well processable into polymer-only microspheres yielding non-porous particles with a smooth surface. No agglomeration was observed. The volume average particle size distribution (D₅₀(vol)) of the microspheres is shown in Table 6.

TABLE 6
Characteristics of polymer-only microspheres
evaluated for their in vitro erosion kinetics

| | | | IV | D₅₀(vol) | C.V. |
|---|---|---|---|---|---|
| Batch # | Polymer grade | RCP | (dl/g) | (μm) | (%) |
| SR16-003 | 100POE40 | 1647 | 0.32 | 51.2 | 28.5% |
| SR16-007 | 10L40-POE40 | 1650 | 0.33 | 41.4 | 30.2% |
| SR16-004 | 50L40-POE40 | 1648 | 0.38 | 37.8 | 22.1% |
| PBD17-056 | 25GL40-POE40 | 1654 | 0.28 | 65.5 | 38.0% |
| PBD17-040 | 50LP10L20-POE40 | 1651 | 0.35 | 65.1 | 21.5% |

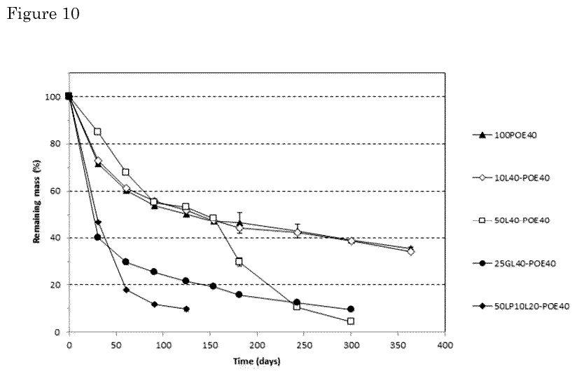

FIG. 10 shows the in vitro erosion of polymer-only microspheres composed of 100POE40, 10L40-POE40-10L40, 50POE40-50L40, 25GL40-5POE40, and 50LP10L20-POE40. 100POE40 based microspheres eroded gradually with approximately 35% of the polymer still remaining after 12 months. The introduction of ˜10 wt. % of poly(D,L-lactide) block (10L40-POE40) did not have any effect on the erosion kinetics, but increasing the poly(D,L-lactide) block content to ˜50 wt. % (50L40-POE40) resulted in complete polymer erosion within 300 days. By replacing the poly(D,L-lactide) block by a poly(D,L-lactide-co-glycolide) block (25GL40-POE40) or by introducing PEG into the poly(D,L-lactide) block (50LP10L20-POE40), erosion of the multiblock copolymer was significantly accelerated.

### Example 8

Ropivacaine-loaded microspheres with a target loading of 50 wt. % were prepared via oil-in-water (O/W) membrane emulsification followed by solvent extraction/evaporation. 1.0 gram of polymer and 1.0 gram of ropivacaine base were dissolved in dichloromethane (DCM) to a final polymer concentration of 15 wt. % to form the dispersed phase (DP). Following filtration over a 0.2 μm polytetrafluoroethylene (PTFE) filter, DP was emulsified with an aqueous solution containing 0.4 wt. % PVA and 5 wt. % NaCl (continuous phase (CP)) via a membrane with 20 μm pores. The formed O/W emulsion was stirred for 2 hours at room temperature followed by 1 hour at 40° C. under an airflow of 5 μl/min to extract and evaporate DCM and harden the microspheres. After completion of solvent evaporation and cooling down to room temperature, the hardened microspheres were collected by filtration and washed three times with 250 ml 0.05 wt. % Tween-80 solution and three times with 250 ml WFI (Water For Injection), after which the microspheres were lyophilised.

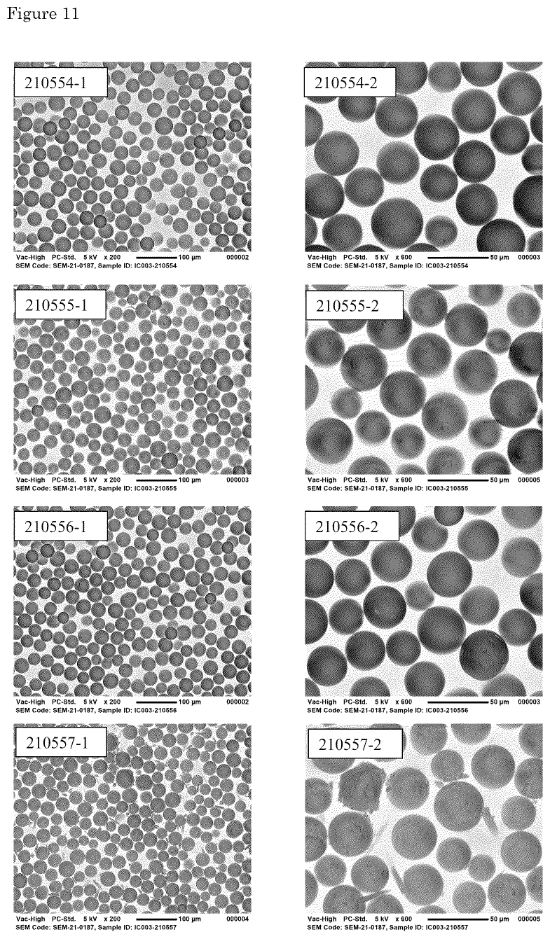

Characterisation of the microspheres by scanning electron microscopy showed that the O/W microencapsulation process yielded spherical microspheres with a smooth surface without any pores (FIG. 11). Crystalline ropivacaine particles were visible on the SEM images of 20LP6L12-POE40 based ropivacaine microspheres indicating that not all ropivacaine was encapsulated in the microspheres (FIG. 11). The average particle size D₅₀(vol) of the ropivacaine loaded microspheres as analysed by laser diffraction varied between 39 and 50 μm (Table 7).

Residual DCM content of the microspheres was determined by gas chromatography with headspace injection and flame-ionisation detection. In brief, 100 mg of sample was dissolved in 5.0 ml of dimethylsulphoxide (DMSO) containing octane as the internal standard. The samples were analysed by GC-Headspace using an Agilent 6850 gas chromatograph, equipped with a Combi-Pal headspace sampler. The calibration range of the method was 55-5500 ppm of DCM in 100 mg sample, using a first order linear regression (weighing factor=1/X). The residual DCM content of the ropivacaine microparticles composed of 20L40-POE40, 50L40-POE40 and 20GL40-POE40 was relatively high (750-1000 ppm), whereas it was significantly lower for 20LP6L12-POE40-based ropivacaine-loaded microspheres (55 ppm).

Ropivacaine content of the microspheres as determined by elemental analysis (Elementar® Micro Cube) varied from 30.5 wt. % for the 20LP6L12-POE40 based ropivacaine microspheres to 41.2 wt. % for the 20L40-POE40 based ropivacaine microspheres (Table 7).

TABLE 7
Characteristics of ropivacaine-loaded microspheres

| | POE- | PSD (μm) * | | | Drug | | DCM |
|---|---|---|---|---|---|---|---|
| IC003 | MBCP | D₁₀ | D₅₀ | D₉₀ | content | EE | content |
| Batch | grade | (vol) | (vol) | (vol) | (wt. %) | (%) | (ppm) |
| 210554 | 20L40- | 32 | 41 | 55 | 41.2 | 83.8 | 899 |
| | POE40 | | | | | | |
| 210555 | 20GL40- | 31 | 40 | 53 | 39.5 | 79.2 | 1018 |
| | POE40 | | | | | | |
| 210556 | 50L40- | 30 | 39 | 51 | 38.2 | 74.1 | 753 |
| | POE40 | | | | | | |
| 210557 | 20LP6L12- | 33 | 50 | 97 | 30.5 | 61.7 | 55 |
| | POE40 | | | | | | |

* PSD means particle size distribution of ropivacaine microspheres with D₁₀(vol) meaning that 10% of the particles have a diameter smaller than the table value, D₅₀(vol) is the volume median particle size (or volume average particle size), and D₉₀(vol) meaning that 90% of the particles have a diameter smaller than the table value.

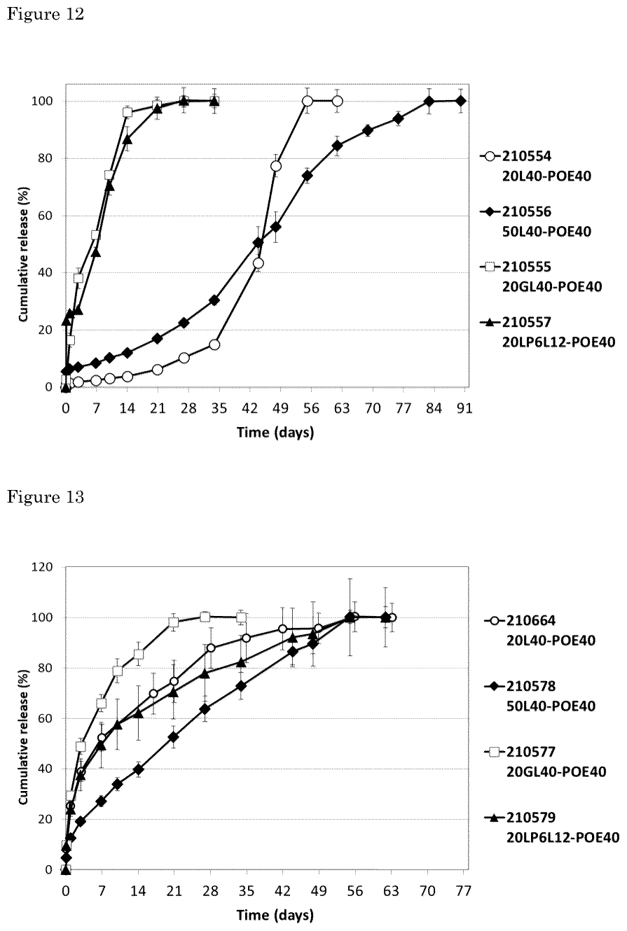

The in vitro release of ropivacaine from the microspheres was determined by incubating 10 mg of ropivacaine microspheres in 45 ml in vitro release buffer (100 mM PO₄ buffer, 0.025% Tween-20, 0.02% NaN₃, 290 mOsm/kg, pH 6.5) at 37° C. At predetermined time points, following centrifugation of the vials, aliquots of 100 μl release buffer were collected. Ropivacaine concentrations in the release buffer were determined via reversed phase ultra-performance liquid chromatography (UPLC) with UV-detection using a Waters Acquity H-Class UPLC system, equipped with a PDA or UV detector, an Acquity BEH C18 column (50×2.1 mm, 1.7 μm), maintained at 40° C. Mobile phase A consisted of a 20 mM phosphate buffer pH 6.5 and acetonitrile at a ratio of 90:10 v/v and 100% of acetonitrile was used as mobile phase B. The mobile phase composition started at 30% B and increased to 70% B in 2 minutes, at a constant flow rate of 0.600 ml/min. Detection was performed at 235 nm. FIG. 12 shows the cumulative release of ropivacaine from the microspheres. 20L40-POE40 and 50L40-POE40 based ropivacaine microspheres released only 5-14% of the encapsulated ropivacaine within the first 14 days. 20GL40-POE40 and 20LP6L12-POE40 based ropivacaine microspheres however, showed complete release of ropivacaine within 14 days.

### Example 9

Ropivacaine-loaded in situ forming implant formulations were prepared using different poly(ortho ester)-based multiblock copolymers by dissolving 1.5 grams of polymer in 2.3 grams of N-methyl-2-pyrrolidone (NMP) and adding 0.5 grams of ropivacaine. The viscosity of the obtained liquid formulations as determined by rheology (TA Instruments AR 2000 rheometer, cone-plate geometry, constant shear at shear rate of 6 l/s) varied from 6 to 21 Pa·s (see Table 8 for more details). The injectability of liquid ropivacaine POE-MBCP based formulations was determined by means of a injectability tester in triplicate. 0.2 ml of the liquid formulation was collected into a 1 ml syringe using a 14 G needle. After removal of air bubbles the 14 G needle was replaced by a 20 G×1″ (0.9 mm×25 mm) needle, after which the syringe with needle was positioned vertically in the tensile tester. The liquid formulation was ejected via the needle at a displacement rate of 100 mm/min and the force displacement curve was recorded. Most of the liquid formulations were well injectable via the 20 G×1″ (0.9 mm×25 mm) needle with ejection forces ranging from 11 to 24 N. The 20GL40-POE40-based formulation, however required significantly higher ejection forces (24-41 N).

Ropivacaine containing POE-MBCP depots were formed in situ by slowly adding 45 ml of buffer (100 mM PO₄ buffer, 0.025% Tween-20, 0.02% NaN₃, 290 mOsm/kg, pH 6.5, 37° C.) to 100 μl of the liquid ropivacaine/polymer/NMP formulations. All formulations showed in situ depot formation without any significant swelling. The in vitro release of ropivacaine from the in situ forming depots was determined by collecting aliquots of 100 μl release buffer at predetermined time points. Ropivacaine concentrations in the release buffer were determined by UPLC as described in Example 8. FIG. 13 shows that the release of ropivacaine from the POE-MBCP based in situ forming implant formulations can be controlled by varying the POE-MBCP composition. Following an initial burst of ˜10%, ropivacaine was gradually released from 50L40-POE40 based depots over a period of 2 months. As expected release from 20GL40-POE40 based depots was significantly faster and complete within 3 weeks due to faster erosion of this polymer as compared to 50L40-POE40. The other two polymers showed intermediate release rates.

TABLE 8
Characteristics of ropivacaine-loaded in situ forming implant
formulations prepared of POE40-based multiblock copolymers

| | | | | | | Maximum |
|---|---|---|---|---|---|---|
| | POE- | Composition (wt. %) | | | | injection |
| IC003 | MBCP | | POE- | | Viscosity | force |
| Batch | grade | Drug | MBCP | NMP | (Pa · s) | (N) * |
| 210664 | 20L40- | 12 | 35 | 53 | 7-13 | 13-18 |
| | POE40 | | | | | |
| 210577 | 20GL40- | 12 | 35 | 53 | 12-17 | 24-41 |
| | POE40 | | | | | |
| 210578 | 50L40- | 12 | 35 | 53 | 6-10 | 13-14 |
| | POE40 | | | | | |
| 210579 | 20LP6L12- | 12 | 35 | 53 | 8-20 | 11-24 |
| | POE40 | | | | | |

* 20 G × 1″ (0.9 mm × 25 mm) needle

### Example 10

Levonorgestrel-loaded in situ forming implant formulations were prepared using different poly(ortho ester)-based multiblock copolymers by dissolving 1.2 g of polymer and 0.05 g of levonorgestrel in 2.2 g NMP. The levonorgestrel-containing liquid formulations were characterised for their viscosity and injectability as described in Example 9.

The viscosity of the liquid levonorgestrel formulations varied from ˜1.6 Pa·s to 4.4 Pa·s (Table 9), which was significantly lower as compared to the liquid ropivacaine formulations in Example 9. All formulations were well injectable via a 21 G×1″ (0.81 mm×25 mm) needle (maximum force 17 N).

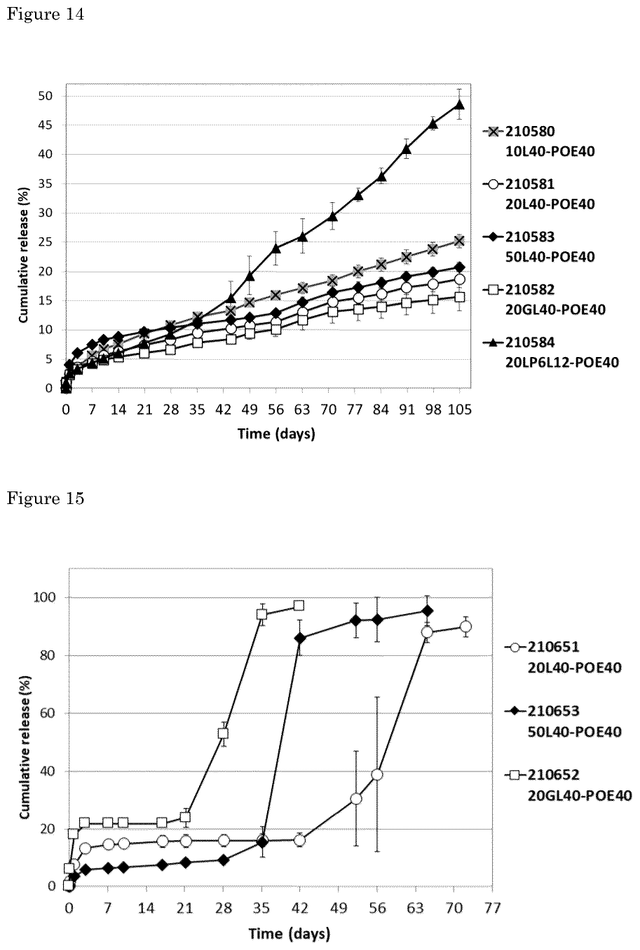

Levonorgestrel containing POE-MBCP depots were formed in situ by slowly adding 14 ml of buffer (100 mM PO₄ buffer, 0.5% SDS, 0.02% NaN₃, 290 mOsm/kg, pH 7.4, 37° C.) to 500 μl of the liquid levonorgestrel/polymer/NMP formulations. All formulations showed in situ depot formation with some swelling of the depots observed after one day. The tubes were placed in a climate chamber thermostated at 37° C. The in vitro release of levonorgestrel from the in situ formed depots was determined by refreshing 13 ml of the release buffer at predetermined time points and analysis of levonorgestrel concentrations in the release buffer via reversed phase UPLC with UV-detection using a Waters Acquity H-Class UPLC system, equipped with a PDA or UV detector, an Acquity BEH C18 column (50×2.1 mm, 1.7 μm), maintained at 40° C. The mobile phase consisted of a water-acetonitrile mixture at an isocratic ratio of 50:50 v/v. The flow rate was set at 0.55 ml/min. Detection was performed at 243 nm. FIG. 14 shows the release of levonorgestrel from the POE based levonorgestrel depots. Very slow release of levonorgestrel was obtained from the depots with only around 15-20% released after 11 weeks, except for the 20LP6L12-POE40 based in situ forming implant which released almost 35% of levonorgestrel.

TABLE 9
Characteristics of levonorgestrel-loaded in
situ forming implant formulations prepared
of various POE40-based multiblock copolymers.

| | | | | | | Maximum |
|---|---|---|---|---|---|---|
| | POE- | Composition (wt. %) | | | | injection |
| | MBCP | | POE- | | Viscosity | force |
| Batch | grade | Drug | MBCP | NMP | (Pa · s) | (N) * |
| 210580 | 10L40- | 1.5 | 35.0 | 63.5 | 1.6-1.7 | 13 |
| | POE40 | | | | | |
| 210581 | 20L40- | 1.5 | 35.0 | 63.5 | 2.4-2.6 | 14 |
| | POE40 | | | | | |
| 210582 | 20GL40- | 1.5 | 35.0 | 63.5 | 4.4 | 14 |
| | POE40 | | | | | |
| 210583 | 50L40- | 1.5 | 35.0 | 63.5 | 1.7-1.8 | 17 |
| | POE40 | | | | | |
| 210584 | 20LP6L12- | 1.5 | 35.2 | 63.3 | 1.9 | 14 |
| | POE40 | | | | | |

* 21 G × 1″ (0.81 mm × 25 mm) needle

### Example 11

Leuprolide-loaded in situ forming implant formulations were prepared using different poly(ortho ester)-based multiblock copolymers by dissolving 0.66 g of polymer in 1.1 g of a mixture of NMP and benzyl benzoate (BB) in a 90/10 weight ratio and adding 0.12 g of leuprolide. The leuprolide-containing liquid formulations were characterised for their viscosity and injectability as described in Example 9. The viscosity of the formulations varied from 1 to 7 Pa·s. All formulations were well injectable via a 20 G×1″ (0.9 mm×25 mm) needle requiring injection forces as low as 8-11 N.

Leuprolide containing POE-MBCP depots were formed in situ by slowly adding 2 ml of buffer (100 mM PO₄ buffer, 0.025% Tween-20, 0.02% NaN₃, 290 mOsm/kg, pH 7.4, 37° C., 0.02% NaN₃, 290 mOsm/kg, pH 7.4) to 100 μl of the liquid leuprolide/polymer/NMP/BB formulations. The in vitro release of leuprolide from the in situ formed depots was determined by refreshing 1.4 ml of the release buffer at predetermined time points. Leuprolide concentrations in the release buffer were determined via reversed phase UPLC with fluorescence-detection using a Waters Acquity H-Class UPLC system, equipped with a fluorescence detector, an Acquity CSH C18 column (50×2.1 mm, 1.7 μm), maintained at 45° C. Mobile phase A consisted of a 37 mM ammonium acetate buffer pH 9.5 and 100% of acetonitrile was used as mobile phase B. The mobile phase composition started at 25% B and increased to 40% B in 1.5 minutes, at a constant flow rate of 0.75 ml/min. Detection was performed at an excitation wavelength of 280 nm and an emission wavelength of 345 nm. FIG. 15 shows the release of leuprolide from the POE-MBCP based in situ forming depots. Leuprolide release from the liquid formulations was characterised by an initial burst release of 5 to 20%, a lag time during which no or hardly any leuprolide was released, after which the release of leuprolide accelerated. The duration of the lag time and onset of accelerated release was dependent on the composition of the POE-MBCP used and occurred earlier for the faster degrading 20GL40-POE40 than for the slower degrading 50L40-POE40 and slowest degrading 20L40-POE40.

TABLE 10
POE-MBCP based in situ forming implant
formulations of leuprolide

| | | | | | | | Maximum |
|---|---|---|---|---|---|---|---|
| | POE- | Composition (wt. %) | | | | | injection |
| IC003 | MBCP | | POE- | | | Viscosity | force |
| Batch | grade | Drug | MBCP | NMP | BB | (Pa · s) | (N) * |
| 210651 | 20L40- | 6 | 33 | 55 | 6 | 6-7 | 8-9 |
| | POE40 | | | | | | |
| 210652 | 20GL40- | 6 | 33 | 55 | 6 | 1 | 11 |
| | POE40 | | | | | | |
| 210653 | 50L40- | 6 | 33 | 55 | 6 | 2-3 | 9 |
| | POE40 | | | | | | |

* 20 G × 1″ (0.9 mm × 25 mm) needle

1. A biodegradable, thermoplastic multiblock copolymer, comprising at least one prepolymer (A) segment and at least one hydrolysable amorphous prepolymer (B) segment, wherein the segments are linked by a multifunctional chain extender,
wherein the prepolymer (A) segment comprises one or more of:
a) one or more hydrolysable linkages, and
b) a water-soluble polymer; and
wherein the hydrolysable amorphous prepolymer (B) segment comprises the following structure: 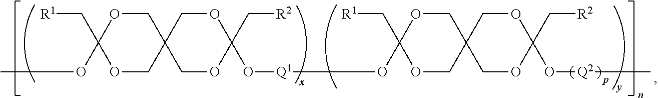
wherein
n is 4-100;
x is 0.25-1 and x+y=1;
p is 0 or 1;
R¹ and R² are independently selected from the group consisting of hydrogen and C₁-C₄ alkyl;
Q¹ is selected from the group consisting of 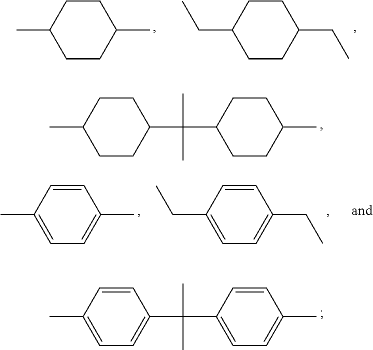
and
Q² is selected from the group consisting of 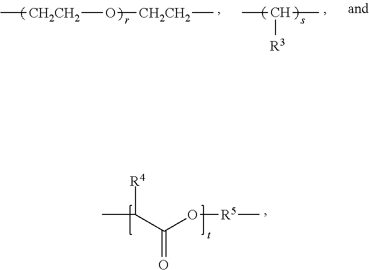
wherein
r is 1-100,
s is 1-12,
t is 1-10,
R³ is selected from the group consisting of hydrogen and C₁-C₆ alkyl,
R⁴ is selected from the group consisting of hydrogen and C₁-C₄ alkyl, and
R⁵ is selected from the group consisting of 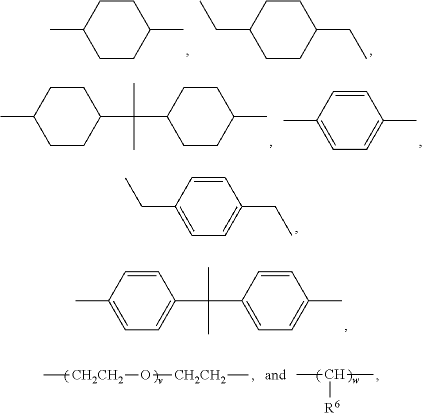
wherein
v is 1-100,
w is 1-12, and
R⁶ is selected from the group consisting of hydrogen and C₁-C₆ alkyl.

2. The biodegradable, thermoplastic multiblock copolymer according to claim 1, wherein R¹ and R² are independently C₁-C₄ alkyl.

3. The biodegradable thermoplastic multiblock copolymer according to claim 2, wherein R¹ and R² are both CH₃, x is 1, and Q¹ is

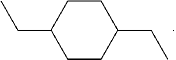

4. The biodegradable, thermoplastic multiblock copolymer of claim 1, wherein said hydrolysable amorphous prepolymer (B) segment has a glass transition temperature T_{g} of 40° C. or more.

5. The biodegradable, thermoplastic multiblock copolymer of claim 1, wherein said prepolymer (A) segment comprises a water-soluble polymer.

6. The biodegradable, thermoplastic multiblock copolymer of claim 1, wherein said water-soluble polymer comprises one or more selected from the group consisting of polyethers, other water-soluble polymers, and copolymers of these polymers.

7. The biodegradable, thermoplastic multiblock copolymer of claim 1, wherein said multifunctional chain extender is a difunctional aliphatic chain extender.

8. The biodegradable, thermoplastic multiblock copolymer of claim 1, wherein prepolymer (A) comprises reaction products of one or more selected from the group consisting of cyclic monomers and non-cyclic monomers.

9. The biodegradable, thermoplastic multiblock copolymer of claim 8, wherein said cyclic monomers are selected from the group consisting of glycolide, lactide, ε-caprolactone, δ-valerolactone, trimethylene carbonate, tetramethylenecarbonate, 1,5-dioxepane-2-one, 1,4-dioxane-2-one (p-dioxanone), cyclic anhydrides, N-carboxyanhydrides of natural amino acids and their derivatives, and morpholine-2,5-diones based cyclic depsipeptides.

10. The biodegradable, thermoplastic multiblock copolymer of claim 8, wherein said non-cyclic monomers are selected from the group consisting of succinic acid, glutaric acid, adipic acid, sebacic acid, lactic acid, glycolic acid, hydroxybutyric acid, natural amino acids and their derivates, ethylene glycol, diethylene glycol, 1,4-butanediol, 1,6-hexanediol, 1,4-butanediamine, and 1,6-hexanediamine.

11. The biodegradable, thermoplastic multiblock copolymer of claim 1, wherein prepolymer (A) has a number average molecular weight (Mₙ) of between 300 and 30,000 g/mol.

12. The biodegradable, thermoplastic multiblock copolymer of claim 1, wherein prepolymer (B) has a number average molecular weight (Mₙ) of 1000 g/mol or more.

13. The biodegradable, thermoplastic multiblock copolymer of claim 1, wherein prepolymer (A) is present in an amount of 1-99% based on total weight of the multiblock copolymer.

14. The biodegradable, thermoplastic multiblock copolymer of claim 1, wherein prepolymer (B) is present in an amount of 1-99% based on total weight of the multiblock copolymer.

15. The biodegradable, thermoplastic multiblock copolymer of claim 1, having an intrinsic viscosity of 0.1 dl/g or more.

16. The biodegradable, thermoplastic multiblock copolymer of claim 1, wherein the prepolymer segments are randomly distributed in the multiblock copolymer.

17. A process for preparing the biodegradable, thermoplastic multiblock copolymer of claim 1, comprising a chain-extension reaction of prepolymer (A) and prepolymer (B) in the presence of a multifunctional chain extender.

18. A composition for delivery of at least one biologically active compound to a host, comprising at least one biologically active compound encapsulated in a matrix, wherein said matrix comprises at least one biodegradable, thermoplastic multiblock copolymer of claim 1.

19. The composition of claim 18, wherein said composition is in the form of one or more selected from the group consisting of microspheres, microparticles, nanoparticles, nanospheres, rods, solid implants, gels, in situ forming implants, coatings, films, sheets, sprays, tubes, membranes, meshes, fibres, and plugs.

20. The composition of claim 18, wherein said composition is in the form of one or more selected from the group consisting of microspheres and microparticles.

21. The composition of claim 18, wherein said composition is in the form of an in situ forming implant, wherein the biologically active compound is dissolved or suspended in a solution of the biodegradable, thermoplastic multiblock copolymer in an acceptable organic solvent, and which solution, following administration into the body, forms in situ a depot by replacement of the organic solvent by aqueous body fluids thereby entrapping the biologically active compound in the biodegradable, thermoplastic multiblock copolymer depot, from which the biologically active compound is subsequently gradually released.

22. The composition of claim 18, wherein said composition is in the form of a solid implant prepared by hot-melt extrusion or injection moulding, and wherein the biologically active compound is incorporated in the biodegradable, thermoplastic multiblock copolymer as a molecular blend or as a dispersion of solid particles.

23. A composition of claim 18, wherein said at least one biologically active compound comprises one or more of a non-peptide non-protein small sized drug, and/or a biologically active polypeptide.

24. A medical device in the form of microspheres, microparticles, nanoparticles, nanospheres, rods, solid implants, gels, in situ forming implants, coatings, films, sheets, sprays, tubes, membranes, meshes, fibres, scaffolds or plugs, wherein said medical device comprises the biodegradable, thermoplastic multiblock copolymer of claim 1.

25. The medical device of claim 24, further comprising at least one biologically active compound encapsulated in the matrix of said biodegradable, thermoplastic multiblock copolymer and being released in a controlled way after insertion in a human or animal.
